SEQUENCE LISTING (12 sequences)

SEQ ID NO: 1 - protein, 120 aa, Artificial Sequence
  Humanized SP34 VH domain from WO2008/119565
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTL

SEQ ID NO: 2 - protein, 125 aa, Artificial Sequence
  BEAT_Ab1 VH domain
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSS

SEQ ID NO: 3 - protein, 125 aa, Artificial Sequence
  BEAT_Ab2 VH domain
EVQLVESGGGLVQPGGSLRLSCAASGFTFNTYAMNWVRQAPGKGLEWVARIRSKYNNYATYYADSVKSRFTISRDDSKNTLYLQMNSLRAEDTAVYYCVRHGNFGNSYVSYFAYWGQGTTVTVSS

SEQ ID NO: 4 - protein, 109 aa, Artificial Sequence
  Humanized SP34 VL domain from WO2008/119565
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 5 - protein, 110 aa, Artificial Sequence
  BEAT_Ab1 VL domain
EIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIK

SEQ ID NO: 6 - protein, 110 aa, Artificial Sequence; identical to SEQ ID NO: 5
  BEAT_Ab2 VL domain

SEQ ID NO: 7 - protein, 214 aa, Artificial Sequence
  BEAT_Ab1 light chain sequence (hum9G7_VL_cK)
DIQMTQSPSSLSASVGDRVTITCQASQDVITSVAWYQQKPGKAPKLLIYSASYRYTGVPSRFSGSGSGTDFTFTISSLQPEDIATYYCQQHYTIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, Artificial Sequence
  BEAT_Ab1 heavy chain sequence (h9G7 1133 BTA LALA FTO (dA))
QVTLKESGPTLVKPTQTLTLTCTFSGLSLSTSGKGVGWIRQPPGKALEWLAHIWWDDDKRYNPALKSRLTITKDTSKNQVVLTMTNMDPVDTATYYCARIELGRSYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 9 - protein, 481 aa, Artificial Sequence
  BEAT_Ab1 scFv-Fc sequence hSP34_H3K21_W100eY/T29E-W91F-L95T (hSP34v.3) scFv_11_BTB_D401Q_LALA
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 10 - protein, 703 aa, Artificial Sequence
  BEAT_Ab2_SF3_scFV_Hc sequence
EVQLVESGGGLVQPGGSLRLSCAASGFTFNTYAMNWVRQAPGKGLEWVARIRSKYNNYATYYADSVKSRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 11 - protein, 214 aa, Artificial Sequence
  BEAT_Ab2_SF3_Lc
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 226 aa, Artificial Sequence
  BEAT_Ab2_SF3_Fc
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPG

(19) United States
(12) Patent Application Publication
(10) Pub. No.: US 2022/0177582 A1
(43) Pub. Date: Jun. 9, 2022

(54) NON-CONSENSUS GLYCOSYLATION OF BISPECIFIC ANTIBODIES

(71) Applicant: ICHNOS SCIENCES SA, La Chaux-de-Fonds (CH)

(72) Inventors: Sachin DUBEY, La Chaux-de-Fonds (CH); Krzysztof BIELIKOWICZ, La Chaux-de-Fonds (CH); Olivier LOPEZ, La Chaux-de-Fonds (CH)

(21) Appl. No.: 17/438,209

(22) PCT Filed: Mar. 13, 2020
(86) PCT No.: PCT/EP2020/056859

§ 371 Date: Sep. 10, 2021

(30) Foreign Application Priority Data
Mar. 13, 2019 (EP) 19162632.4

(51) Int. Cl. C07K 16/28

(52) U.S. Cl. CPC C07K 16/2809; C07K 2317/41; C07K 2317/622

(57) ABSTRACT

The present invention relates to antibodies or antibody fragments comprising non-consensus glycosylation. The present invention also relates to methods to remove and measure said non-consensus glycosylation.

### TECHNICAL FIELD

The present invention relates to antibodies or antibody fragments comprising non-consensus glycosylation. The present invention also relates to methods to remove and measure said non-consensus glycosylation.

### BACKGROUND

Protein glycosylation is a common post-translational modification which affects the folding and conformation of a protein and therefore its activity and function. When proteins, such as antibodies, are used for therapeutic purposes, it is necessary to take into account the role of glycosylation since it may impact their efficacy, safety, pharmacokinetics and pharmacodynamics (L. Liu, Journal of Pharmaceutical Sciences, June 2015, Vol 104, Issue 6, pages 1866-1884).

Depending from their class and type, antibodies present different glycosylation characteristics. In general, antibodies have a conserved N-linked glycan attached to the fragment crystallizable (Fc) asparagine 297 of each heavy chain. Since the shape of the Fc region defines the capacity of the antibody to interact with innate immune Fc receptors, such glycosylation affects the antibody functionality (MF. Jennewein et al. Trends in Immunology May 2017, Vol 38, Issue 38, pages 358-372). Approximately 20% of the antibody contain a second N-linked glycosylation site in their variable region. Both sites are located on the heavy chain. N-glycans are highly heterogeneous due to the high number of different sugar moieties and the multitude of possible linkages (Higel et al. European Journal of Pharmaceutics and Biopharmaceutics, March 2016, Vol 100, pages 91-100). Additionally, antibodies can present O-linked glycans, which have typically a shorter structure than N-linked glycans and they are present in the hinge region between the Fragment antigen-binding (Fab) and Fc portion of the heavy chain of some Ig (IgA1 and IgD). Moreover, antibodies may have unusual attachment sites for glycosylation at non-consensus sites (Spearman et al. Antibody Expression and Production, May 2011, Chapter 12, pages 251-292) which further affect their folding and binding capacity.

Understanding the glycosylation properties of antibodies used as therapeutics is critical for better characterizing their structures and properties, and to assure their safety in patients and their proper activity.

### DESCRIPTION

The present invention relates to antibodies or antibody fragments comprising non-consensus glycosylation. The present invention also relates to methods to remove and measure said non-consensus glycosylation.

In particular the present invention relates to a purified antibody or fragment thereof comprising a single chain variable fragment which is glycosylated in at least one non-consensus glycosylation site. More in particular the purified antibody or fragment thereof binds CD3.

In an aspect of the present invention the single chain variable fragment of the disclosed purified antibody or fragment thereof comprises a variable heavy chain of amino acid sequence selected from the group comprising SEQ ID NOs: 1, 2 and 3, and conservative modifications thereof, and a variable light chain of amino acid sequence selected from the group comprising SEQ ID NOs: 4, 5 and 6, and conservative modifications thereof.

In another aspect, the non-consensus glycosylation site of the purified antibody or fragment thereof of the present invention is a QGT motif. More in particular non-consensus glycosylation site of the purified antibody or fragment thereof of the present invention is glycosylated with a glycan selected from the group comprising G0F, G1G, G1FS, G2FS and GSFS2.

In an even more particular aspect, the present invention discloses a purified antibody or fragment thereof wherein a single chain variable fragment is glycosylated with a glycan selected from the group comprising G2FS and G2FS2, in a QGT motif at position 117-119 of SEQ ID NO: 2.

In another aspect, the purified antibody or fragment thereof comprising the amino acid sequence of SEQ ID NOs: 7, 8 and 9 or the amino acid sequence of SEQ ID NOs: 10, 11 and 12.

The present invention also relates to a deglycosylated protein obtained by a process comprising a step of incubation of a protein glycosylated in at least one non-consensus glycosylation site with rapid PNGase enzyme in native conditions.

The present invention also relates to a method for quantifying a protein glycosylated in at least one non-consensus glycosylation site which is comprised in a purified protein mixture, comprising the step of subjecting said purified protein mixture to reduced or non-reduced CE-SDS analysis.

The present invention also relates to a method to generate a material enriched with a purified protein variant of interest, wherein said purified protein variant of interest is comprised in a purified protein mixture, comprising the steps of:
(a) Subjecting said purified protein material to chromatography;
(b) Identifying the peak comprising said purified protein variant of interest;
(c) Eluate said peak in at least two fractions;
(d) Identifying the fraction(s) containing said protein variant of interest by CE-SDS;
(e) Repeat steps (a) to (d) by subjecting the fraction(s) identified in (d) to the chromatography step in (a) until the desired percentage of the purified protein variant of interest is reached, wherein said desired percentage is between about 40% to about 90%.

More in particular said chromatography is selected from the group comprising size exclusion chromatography (SEC), ions exchange chromatography, anion exchange chromatography (AEX), cation exchange chromatography (CEX), affinity chromatography, hydrophobic interaction chromatography (HIC), reverse phase chromatography (RP), high-pressure liquid chromatography (HPLC) chromatography including SE-HPLC, CEX-HPLC, AEX-HPLC, HIC-HPLC, RP-HPLC. Specifically, said chromatography is SE-HPLC or CEX-H PLC.

In an aspect of the present invention, the protein of the disclosed methods is an antibody or an antibody fragment thereof; particularly an antibody fragment thereof is the antibody or an antibody fragment thereof of claims 1 to 8.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of cell and tissue culture, molecular biology, and protein and oligo- or polynucleotide chemistry, laboratory procedures and techniques of analytical chemistry, synthetic organic chemistry, and medicinal and pharmaceutical chemistry described herein are those well-known and commonly used in the art.

Disclosed by the present invention is a purified antibody or antibody fragment thereof which is glycosylated in at least a glycosylation site other than a consensus glycosylation sites.

Glycosylation is the process by which a carbohydrate is covalently attached to a target macromolecule, such as a protein, e.g. an antibody or antibody fragment. Protein glycosylation is a co-translational and/or post-translational modification affecting the folding, conformation, activity and interaction of said protein. In the present invention the terms “carbohydrate” and “glycan” are used interchangeably. Several classes of glycans exist, including N-linked glycans, O-linked glycans. N-linked glycans are attached to a nitrogen of asparagine or arginine side-chains of a protein. O-linked glycans attached to the hydroxyl oxygen of serine, threonine, tyrosine, hydroxylysine, or hydroxyproline side-chains of a protein. This type of glycosylation involve the linkage between the monosaccharide N-Acetylgalactosamine and the amino acid Serine or Threonine.

Monoclonal antibodies are glycoproteins comprising two conserved N-glycosylation sites on the fragment crystallizable region (Fc), and optionally glycosylation sites on the antibody binding fragment (Fab. Glycosylation may take place on consensus glycosylation sites. In the present application the terms “consensus glycosylation site”, “consensus site”, “consensus glycosylation motif”, “consensus motif”, “consensus glycosylation sequence”, “consensus sequence” are used interchangeably to indicate an amino acid motif known to be gycosylated. Consensus glycosylation sites for N-glycosylation comprise the following motifs: Asn-Xaa-Ser, Asn-Xaa-Thr and Asn-Xaa-Cys, wherein Xaa is any amino acid. It has been shown that the presence of proline between Asn and Ser/Thr will inhibit N-glycosylation.

Disclosed by the present invention is a purified antibody or antibody fragment thereof which is glycosylated in at least one non-consensus glycosylation site. As used herein the term “non-consensus glycosylation site” refers to an amino acid motif other than the consensus glycosylation motif that can be glycosylated. In one embodiment, the non-consensus glycosylation site on which the purified antibody or antibody fragment thereof of the present invention is glycosylated is a Gln-Gly-Thr (QGT) motif. In a more specific embodiment the purified antibody or antibody fragment thereof of the present invention is glycosylated in a non-consensus glycosylation site with a glycan selected from the group comprising G0F, G1G, G1FS, G2FS and GSFS2.

In the present invention, the term “antibody” and the term “immunoglobulin” are used interchangeably. The term “antibody” as referred to herein, includes the full-length antibody and antibody fragments. Antibodies are glycoproteins produced by plasma cells that play a role in the immune response by recognizing and inactivating antigen molecules. In mammals, five classes of immunoglobulins are produced: IgM, IgD, IgG, IgA and IgE. In the native form, immunoglobulins exist as one or more copies of a Y-shaped unit composed of four polypeptide chains: two identical heavy (H) chains and two identical light (L) chains. Covalent disulfide bonds and non-covalent interactions allow inter-chain connections; particularly heavy chains are linked to each other, while each light chain pairs with a heavy chain. Both heavy chain and light chain comprise an N-terminal variable (V) region and a C-terminal constant (C) region. In the heavy chain, the variable region is composed of one variable domain (VH), and the constant region is composed of three or four constant domains (CH1, CH2, CH3 and CH4), depending on the antibody class; while the light chain comprises a variable domain (VL) and a single constant domain (CL). The variable regions contain three regions of hypervariability, termed complementarity determining regions (CDRs). These form the antigen binding site and confer specificity to the antibody. CDRs are situated between four more conserved regions, termed framework regions (FRs) that define the position of the CDRs. Antigen binding is facilitated by flexibility of the domains position; for instance, immunoglobulin containing three constant heavy domains present a spacer between CH1 and CH2, called “hinge region” that allows movement for the interaction with the target. Starting from an antibody in its intact, native form, enzymatic digestion can lead to the generation of antibody fragments. For example, the incubation of an IgG with the endopeptidase papain, leads to the disruption of peptide bonds in the hinge region and to the consequent production of three fragments: two antibody binding (Fab) fragments, each capable of antigen binding, and a cristallizable fragment (Fc). The fragment crystallizable region is the region of an antibody which interacts with cell surface receptors called Fc receptors and some proteins of the complement system. This allows antibodies to activate the immune system. The Fc region of IgGs bear a highly conserved N-glycosylation site which is essential for Fc receptor-mediated activity. The N-glycans attached to this site are predominantly core-fucosylated diantennary structures of the complex type. Digestion by pepsin instead yields one large fragment, F(ab′)2, composed by two Fab units linked by disulfide bonds, and many small fragments resulting from the degradation of the Fc region. Depending on their nature, antibodies and antibody fragments can be monomeric or multimeric, monovalent or multivalent, monospecific or multispecific.

The term “antibody fragments” as used herein, includes one or more portion(s) of a full-length antibody. Non limiting examples of antibody fragments include: (i) the fragment crystallizable (Fc) composed by two constant heavy chain fragments which consist of CH2 and CH3 domains, in IgA, IgD and IgG, and of CH2, CH3 and CH4 domains, in IgE and IgM, and which are paired by disulfide bonds and non-covalent interactions; (ii) the fragment antigen binding (Fab), consisting of VL, CL and VH, CH1 connected by disulfide bonds; (iii) Fab′, consisting of VL, CL and VH, CH1 connected by disulfide bonds, and of one or more cysteine residues from the hinge region; (iv) Fab′-SH, which is a Fab′ fragment in which the cysteine residues contain a free sulfhydryl group; (v) F(ab′)2 consisting of two Fab fragments connected at the hinge region by a disulfides bond; (vi) the variable fragments (Fv), consisting of VL and VH chains, paired together by non-covalent interactions; (vii) the single chain variable fragments (scFv), consisting of VL and VH chains paired together by a linker; (ix) the bispecific single chain Fv dimers, (x) the scFv-Fc fragment; (xi) a Fd fragment consisting of the VH and CH1 domains; (xii) the single domain antibody, dAb, consisting of a VH domain or a VL domain; (xiii) diabodies, consisting of two scFv fragments in which VH and VL domains are connected by a short peptide that prevent their pairing in the same chain and allows the non-covalent dimerization of the two scFvs; (xiv) the trivalent 10 triabodies, where three scFv, with VH and VL domains connected by a short peptide, form a trimer. (xv) half-IgG, comprising a single heavy chain and a single variable chain.

The term “valence” as used herein, refers to the number of binding sites in the antibody. An antibody that has more than one valence is called multivalent; non-limiting examples of multivalent antibodies are: bivalent antibody, characterized by two biding sites, trivalent antibody, characterized by three binding sites, and tetravalent antibody, characterized by four binding sites.

The term “monospecific antibody” as used herein, refers to any antibody or fragment having one or more binding sites, all binding the same epitope.

The term “multispecific antibody” as used herein, refers to any antibody or fragment having more than one binding site that can bind different epitopes of the same antigen, or different antigens. A non-limiting example of multispecific antibodies are bispecific antibody.

The term “bispecific antibody” refers to any antibody having two binding sites that can bind two different epitopes of the same antigen, or two different antigens.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen” as used herein, refers to any molecule to which an antibody can specifically bind. Examples of antigens include polypeptides, proteins, polysaccharides and lipid molecules. In the antigen one or more epitopes can be present. The term “epitope” or “antigenic determinant” as used herein, refers to the portion of the antigen that makes the direct chemical interaction with the antibody.

As used herein, the term “epitope” includes any protein determinant capable of specific binding to/by an immunoglobulin or fragment thereof, or a T-cell receptor. The term “epitope” includes any protein determinant capable of specific binding to/by an immunoglobulin or T-cell receptor. Epitopic determinants usually consist of chemically active surface groupings of molecules such as amino acids or sugar side chains and usually have specific three dimensional structural characteristics, as well as specific charge characteristics.

In a particular embodiment, the present application discloses a purified antibody or fragment thereof comprising a single chain variable fragment (scFv) which is glycosylated in at least one non-consensus glycosylation site. In a more specific embodiment, the scFv comprises a variable heavy chain of amino acid sequence selected from the group comprising SEQ ID NOs: 1, 2 and 3, and conservative modifications thereof, and a variable light chain of amino acid sequence selected from the group comprising SEQ ID NOs: 4, 5 and 6, and conservative modifications thereof.

In one aspect of the present invention, the purified antibody or fragment thereof is a monoclonal antibody, more particularly a bispecific monoclonal antibody. In a more particular aspect, the purified antibody or fragment thereof of the present invention bids CD3.

In the present invention, the bispecific antibody may be generated by BEAT® technology (WO2012131555). In one embodiment, the bispecific antibody provide by the present invention binds to epitopes upon CD3E and CD38 (SEQ ID NOs: 7 to 9). In particular BEAT_Ab1 was designed to simultaneously engage the CD3 molecule on T cells and the CD38 antigen on multiple myeloma cells and thus bridge cytotoxic T cells to multiple myeloma tumor cells, thereby killing the bound target cells. This process is described as redirected killing or lysis. In another embodiment, the monoclonal bispecific antibody is BEAT_Ab2 (SEQ ID NOs: 10 to 12), which binds to CD3 and EGFR, known to be a target in different types of cancers, including colorectal cancer.

As discussed herein, minor variations in the amino acid sequences of antibodies or immunoglobulin molecules are contemplated as being encompassed by the present disclosure, providing that the variations in the amino acid sequence maintain at least 75%, for example, at least 80%, 90%, 95%, or 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family.

In a particular embodiment, the purified antibody or fragment thereof of the present application comprising a scFv is glycosylated with a glycan selected from the group comprising G2FS and G2FS2, in a QGT motif at position 117-119 of SEQ ID NO: 2.

Also disclosed by the present invention is a deglycosylated protein, such as an antibody or antibody fragment thereof, obtained by a process comprising a step of incubation of a protein glycosylated in at least one non-consensus glycosylation site with rapid PNGase enzyme in native conditions.

Additionally, the present invention discloses a method for quantifying a protein, such as an antibody or antibody fragment thereof, glycosylated in at least one non-consensus glycosylation site and which is comprised in a purified protein mixture comprising the step of subjecting said purified protein mixture to reduced or non-reduced capillary gel electrophoresis (CE-SDS) which allows the separation of molecules based on their size.

Disclosed herein is also a method to generate a material enriched with a purified protein variant of interest, such as an antibody or antibody fragment thereof, wherein said purified protein variant of interest is comprised in a purified protein mixture, comprising the steps of:
(a) Subjecting said purified protein material to chromatography;
(b) Identifying the peak comprising said purified protein variant of interest;
(c) Eluate said peak in at least two fractions;
(d) Identifying the fraction(s) containing said protein variant of interest by CE-SDS;
(e) Repeat steps (a) to (d) by subjecting the fraction(s) identified in (d) to the chromatography step in (a) until the desired percentage of the purified protein variant of interest is reached, wherein said desired percentage is between about 40% to about 90%.

In a more particular embodiment said chromatography is selected from size exclusion chromatography (SEC), ions exchange chromatography, anion exchange chromatography (AEX), cation exchange chromatography (CEX), affinity chromatography, hydrophobic interaction chromatography (HIC), reverse phase chromatography (RP), high-pressure liquid chromatography (HPLC) chromatography including SE-HPLC, CEX-HPLC, AEX-HPLC, HIC-HPLC, RP-HPLC. More specifically, said chromatography is SE-HPLC or CEX-HPLC.

Size exclusion chromatography is a chromatographic method in which molecules in solution are separated by their size. The chromatography column is packed with fine porous beads composed of different kind of polymers. Due to the pore of the beads, small compound and small molecules are retained longer within the column and will be eluted later while larger molecule will be eluted first. Ion exchange chromatography is process that separates ions and polar molecules based on their affinity to the ion exchanger. In order to work the conditions used needs to be out of the isoelectric point of a protein to get charged proteins. Cation exchange chromatography is used when the molecule of interest is positively charged because the pH for chromatography is less than the pi. The stationary phase is negatively charged and positively charged molecules are loaded to be attracted to it.

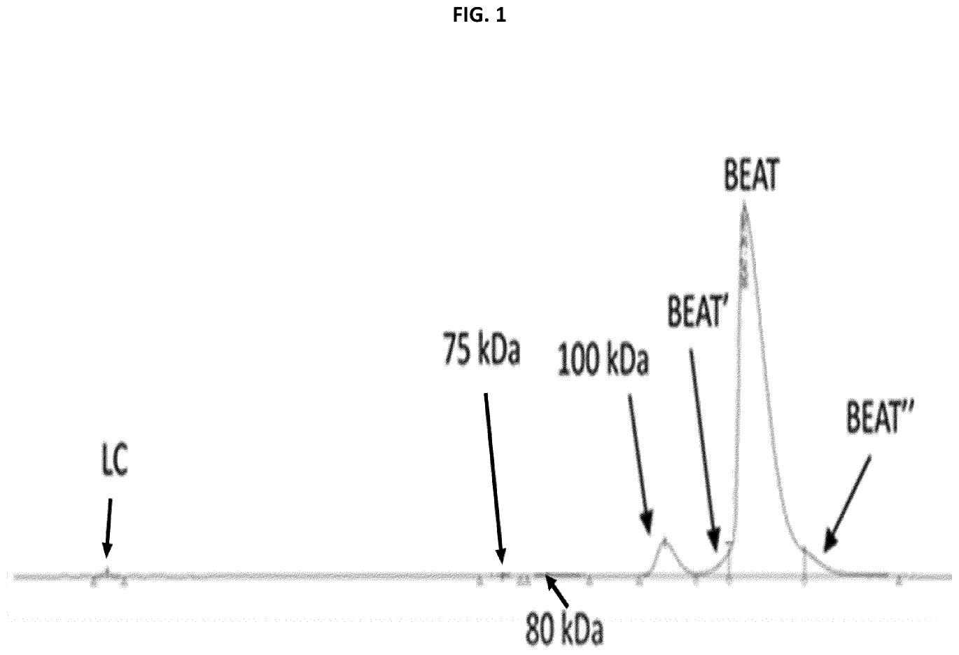

FIG. 1: BEAT_Ab1 BDS—non-reducing CE-SDS profile

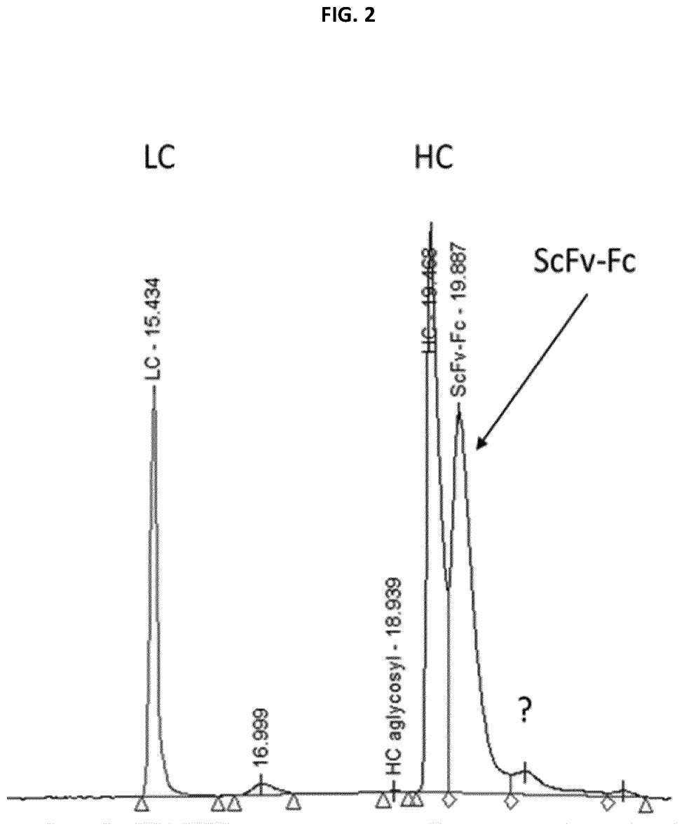

FIG. 2: BEAT_Ab1 BDS—reducing CE-SDS profile

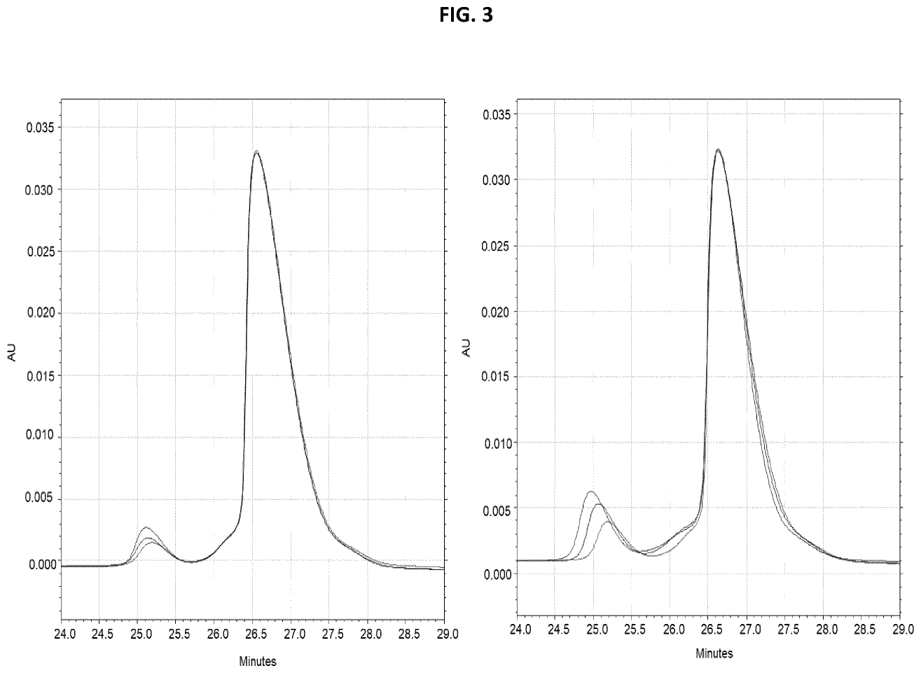

FIG. 3: CE-SDS overlay of BEAT_AB1 BDS denatured at different time and temperature

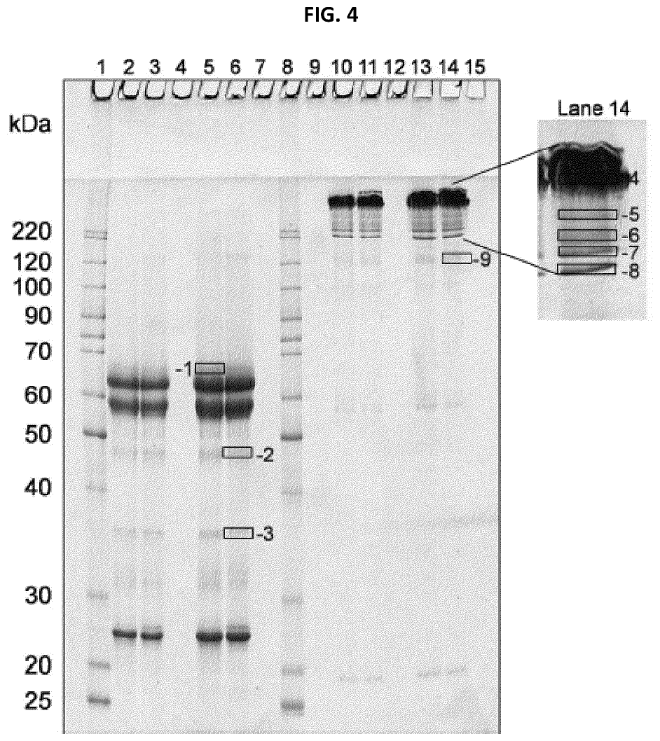

FIG. 4: SDS_PAGE gel image with annotation of the spots (1091-1 to 1091-9) selected for MS/MS identification.

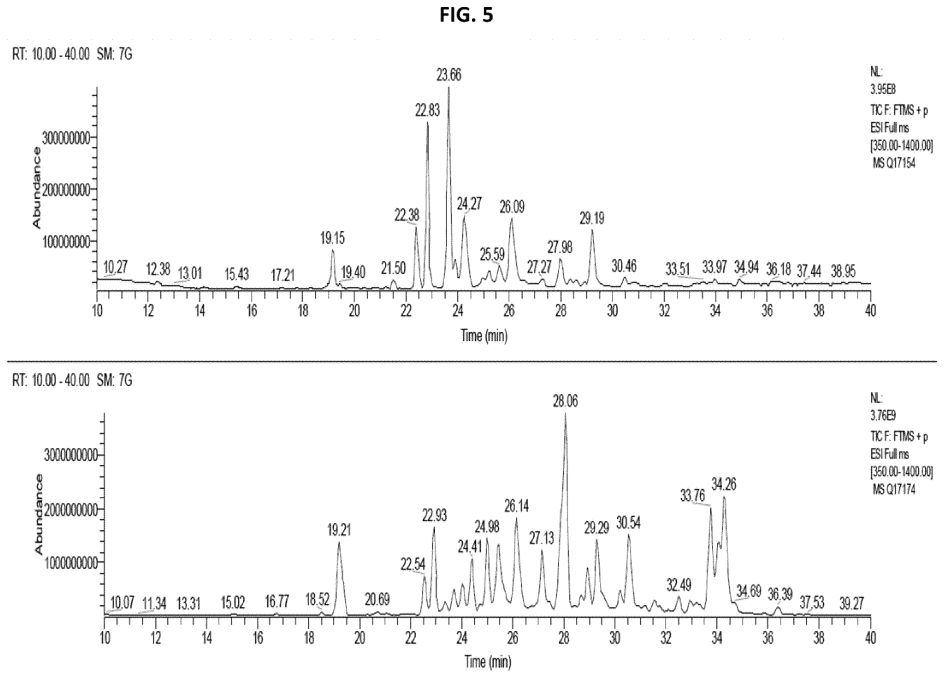

FIG. 5: Total Ion Current (TIC) profile for gel band 1 and 4

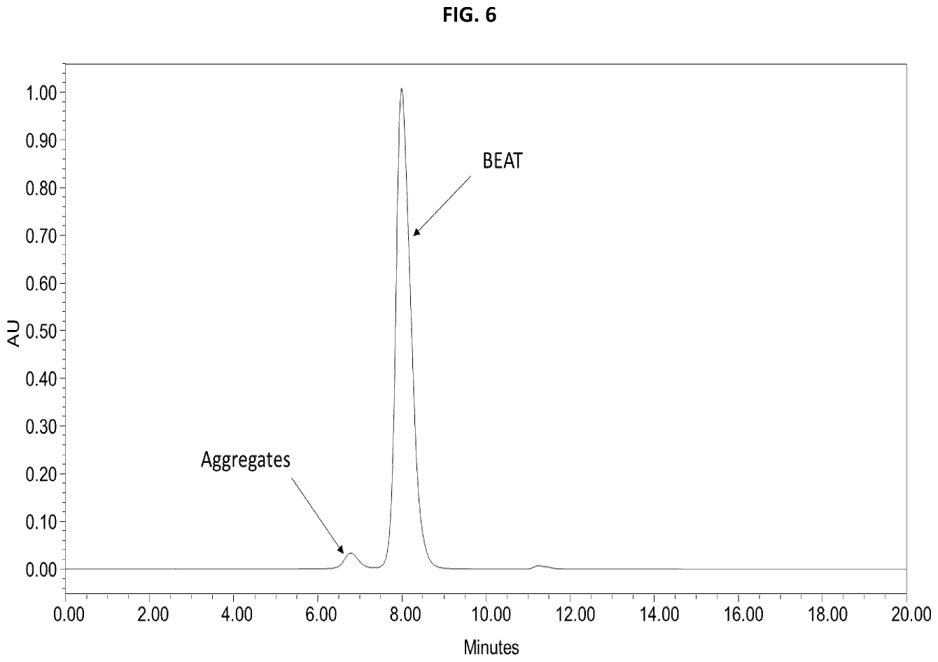

FIG. 6: SE-HPLC profile of BEAT_Ab1_BDS

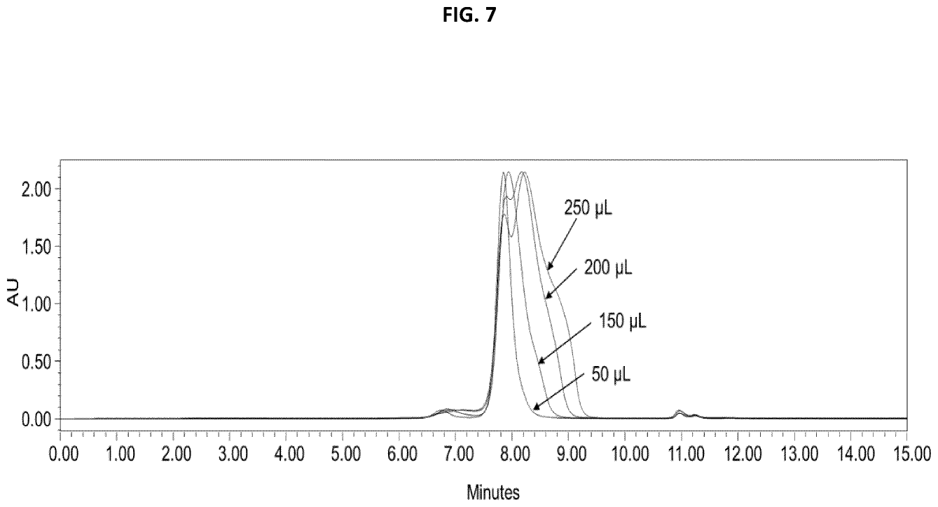

FIG. 7: SE-HPLC chromatogram for the test of volume injected on column

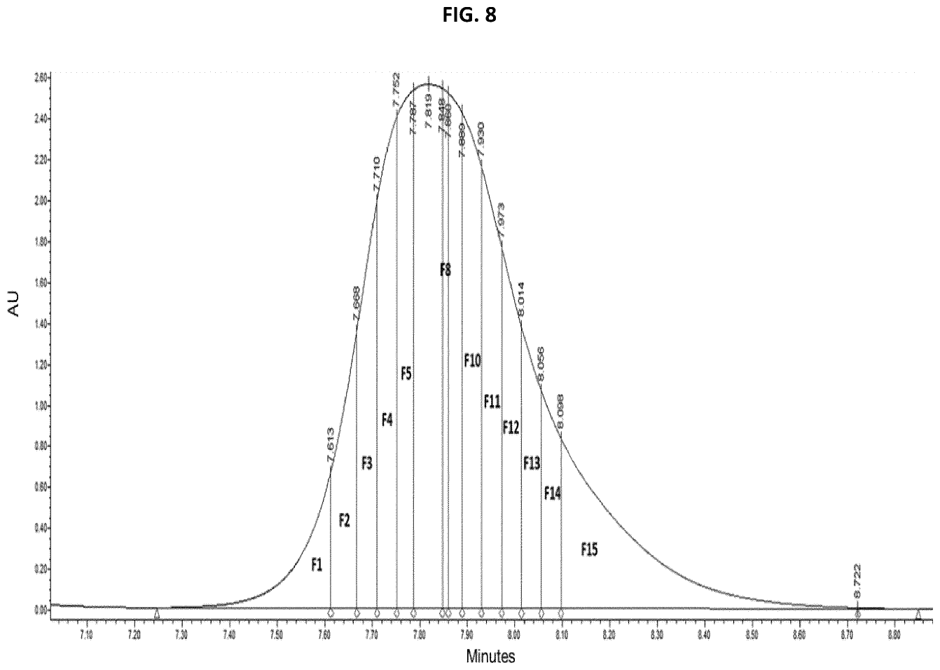

FIG. 8: SE-HPLC chromatogram of BEAT_Ab1_BDS showing the 12 fractions which have been successfully collected and analyzed on non-reducing CE-SDS.

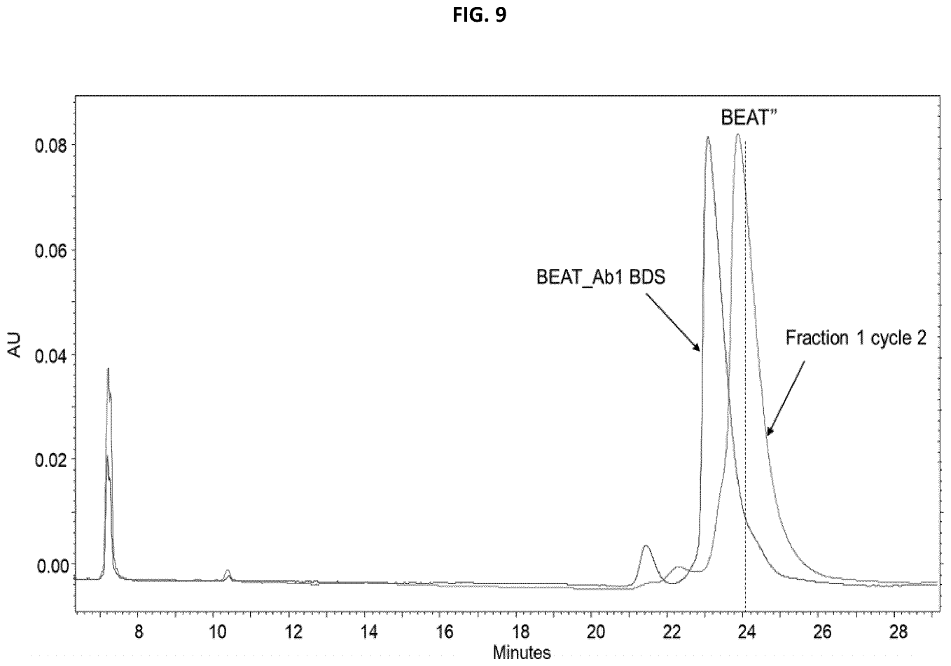

FIG. 9: Non-reducing CE-SDS profile of final enriched BEAT″ material

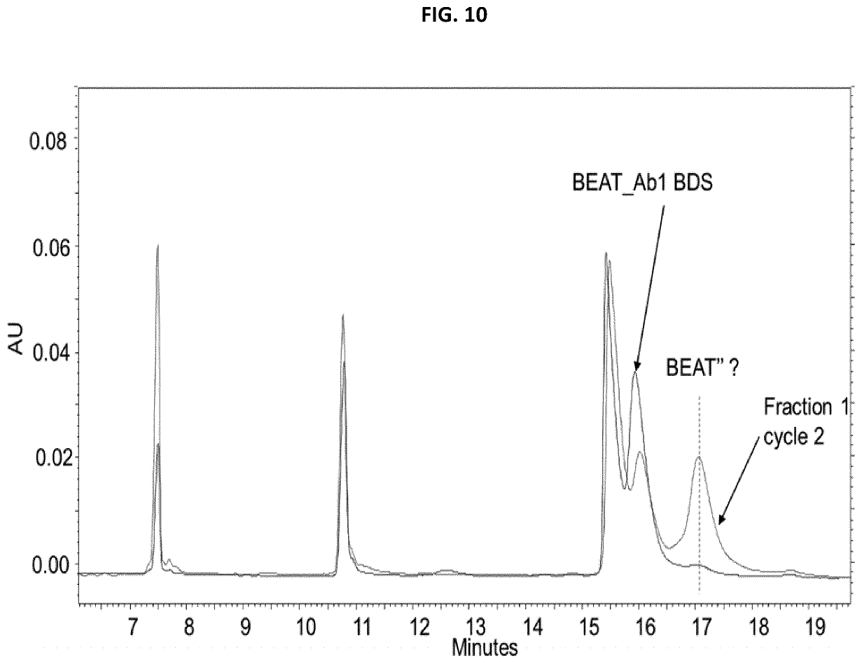

FIG. 10: Reducing CE-SDS profile of final enriched BEAT″ material

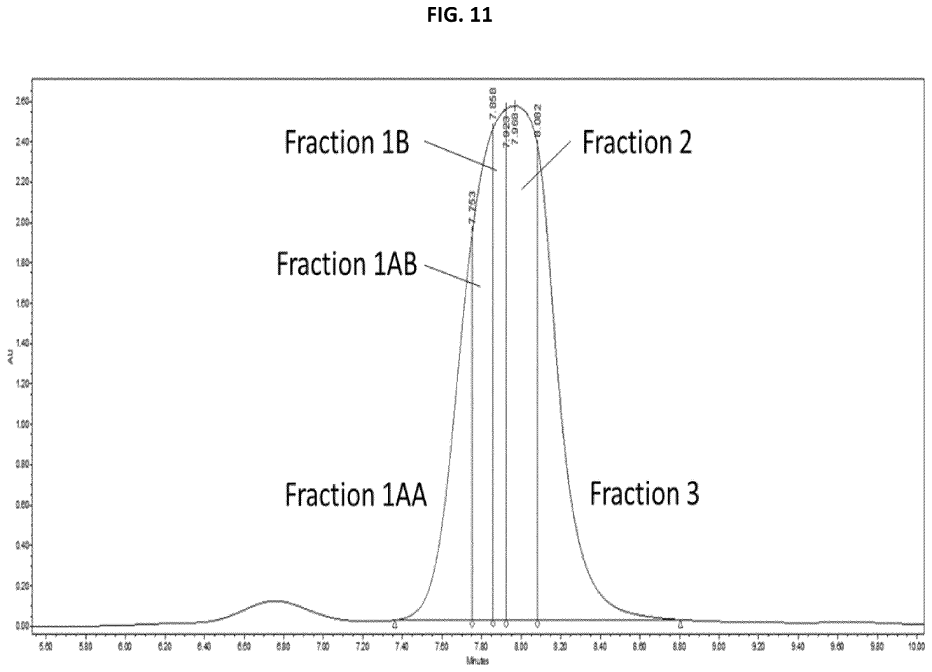

FIG. 11: SE-HPLC monomer fractions during second enrichment experiment.

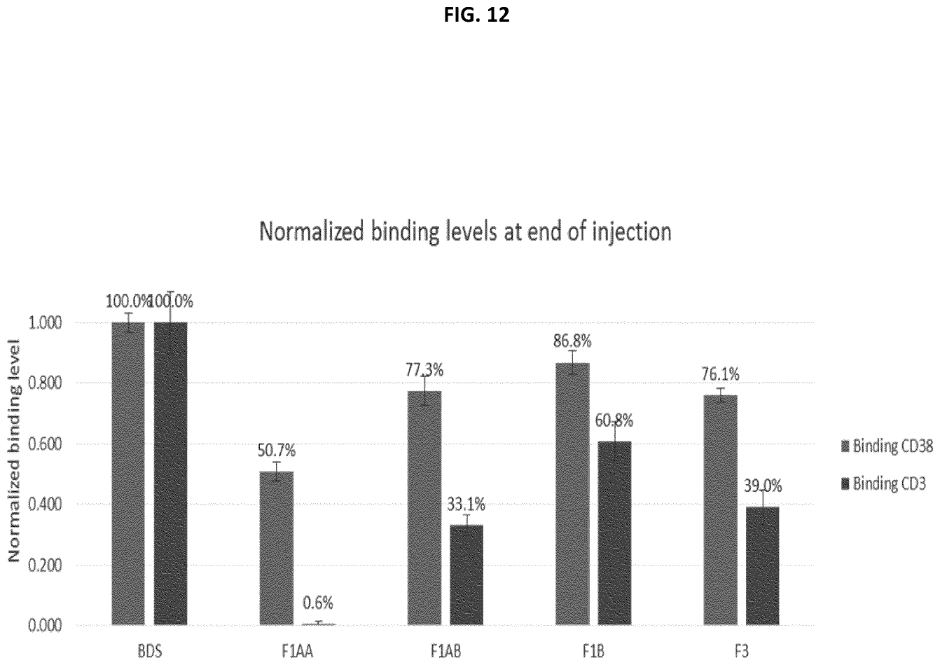

FIG. 12: SPR (Biacore) binding results for SEC-enriched fractions.

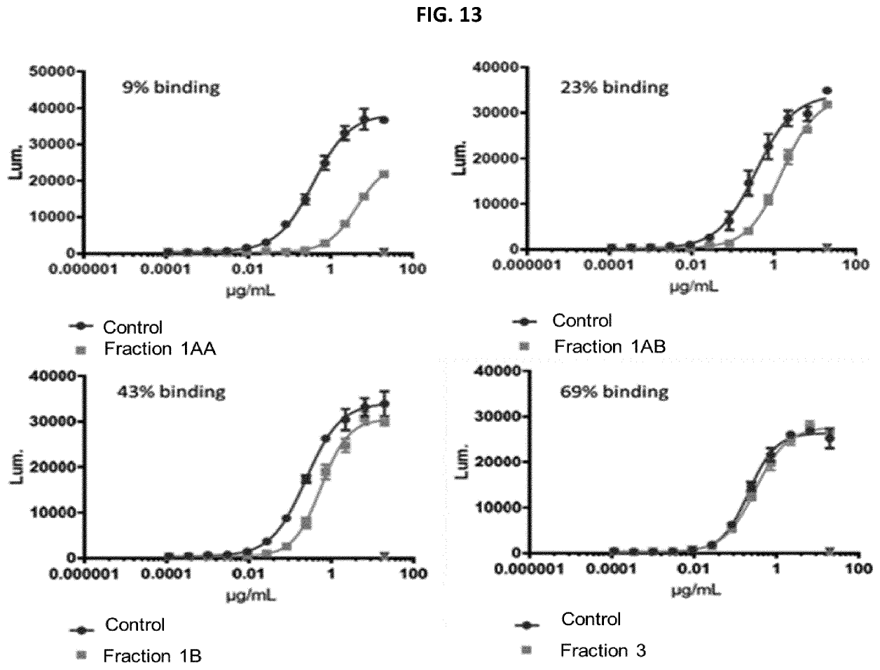

FIG. 13: Binding curves from potency assay of Fraction 1AA, Fraction 1AB, Fraction 1B, Fraction 3.

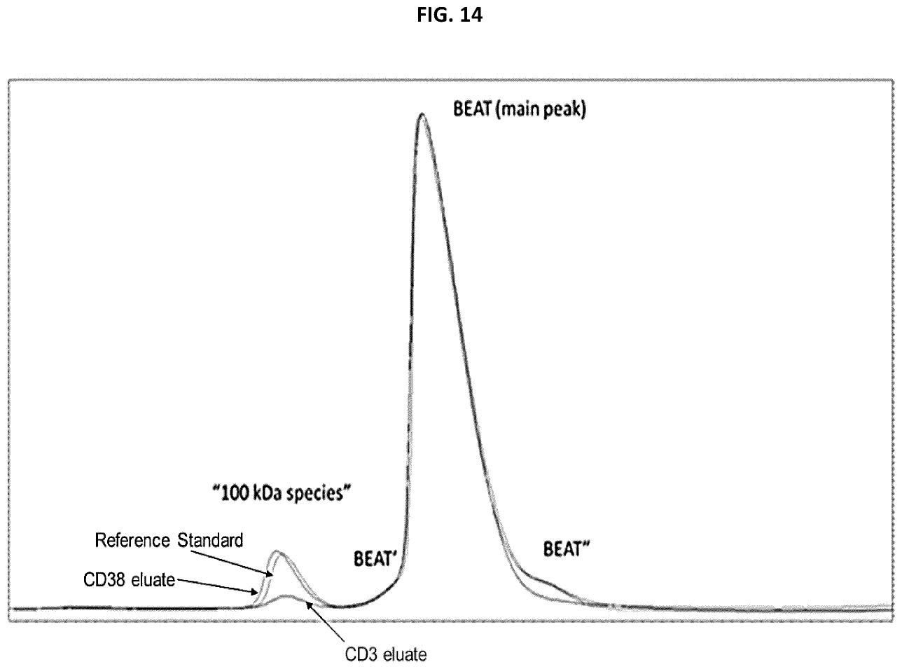

FIG. 14: Overlay of non-reduced CE-SDS profiles of affinity purification eluates

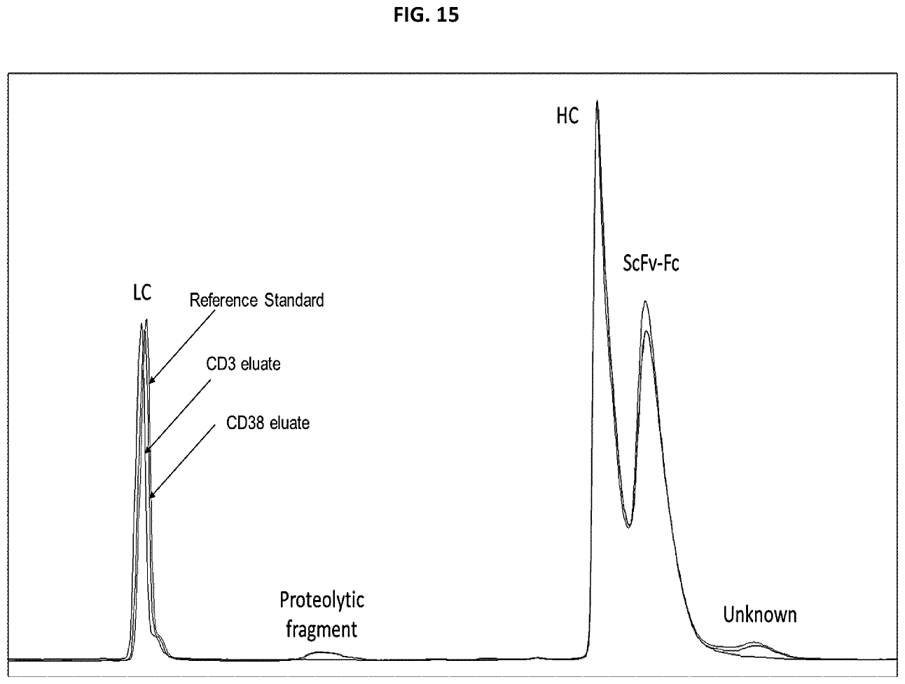

FIG. 15: Overlay of reduced CE-SDS profiles of affinity purification eluates

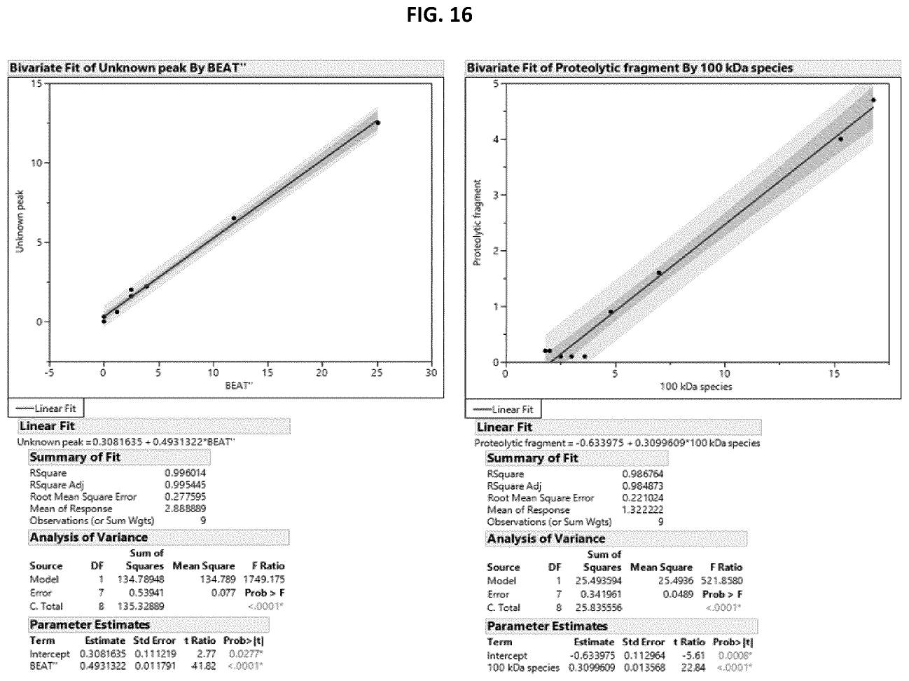

FIG. 16: Linear fit of BEAT″ and “Unknown Peak” (left), and “100 kDa species” and “Proteolytic fragment” (right).

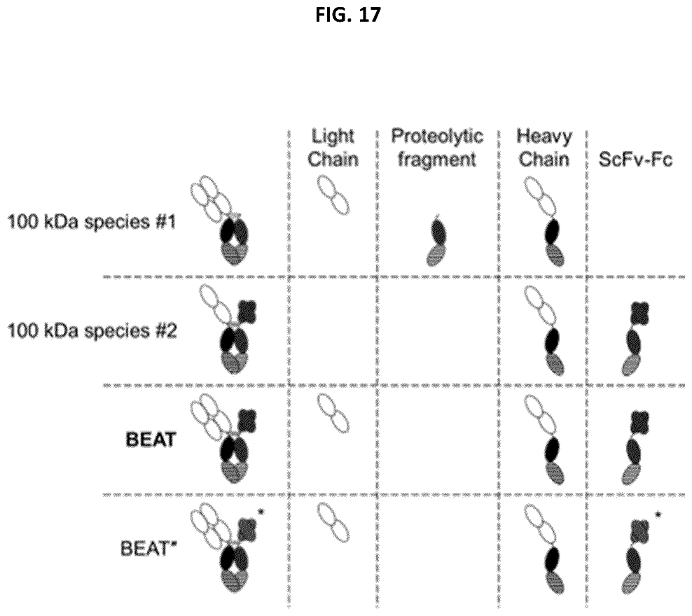

FIG. 17: Proposed structures of peaks observed on non-reducing (vertical) and reducing (horizontal) CE-SDS of BEAT_Ab1.

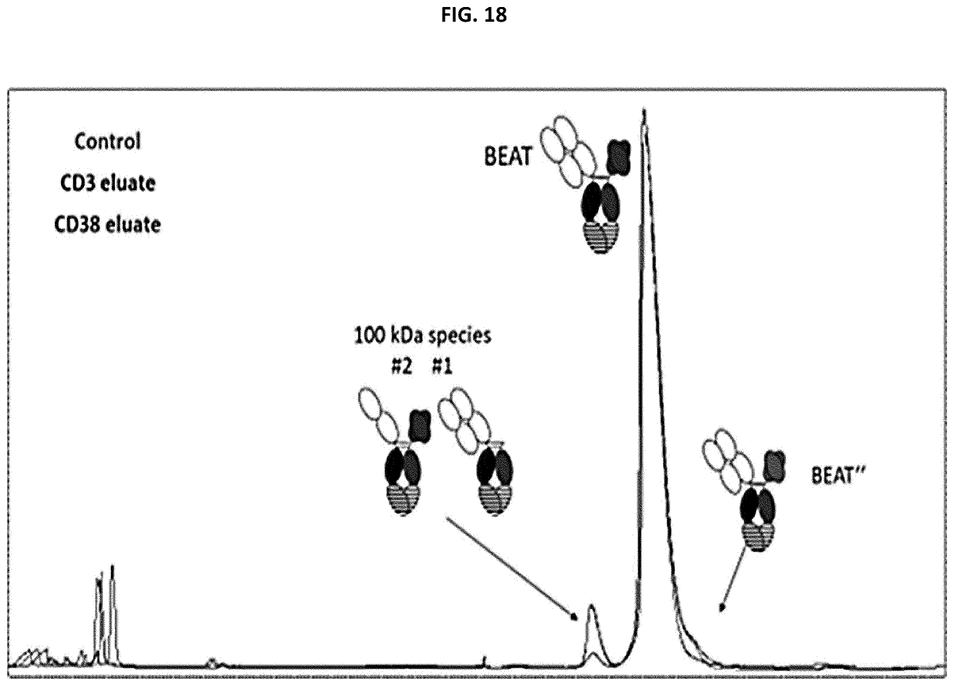

FIG. 18: BEAT_Ab1 non-reduced CE-SDS with proposed structures.

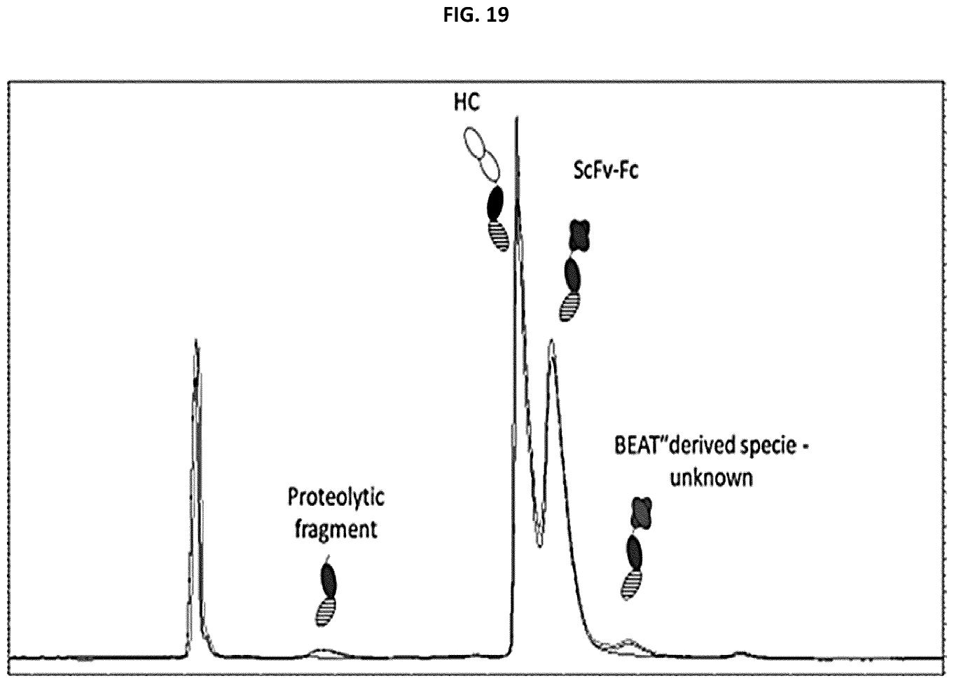

FIG. 19: BEAT_Ab1 reduced CE-SDS with proposed structures.

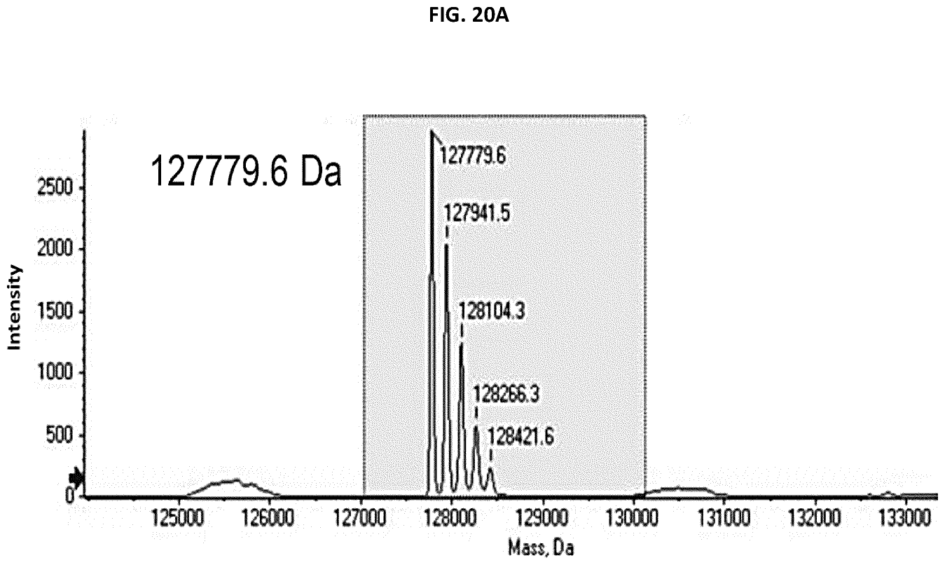

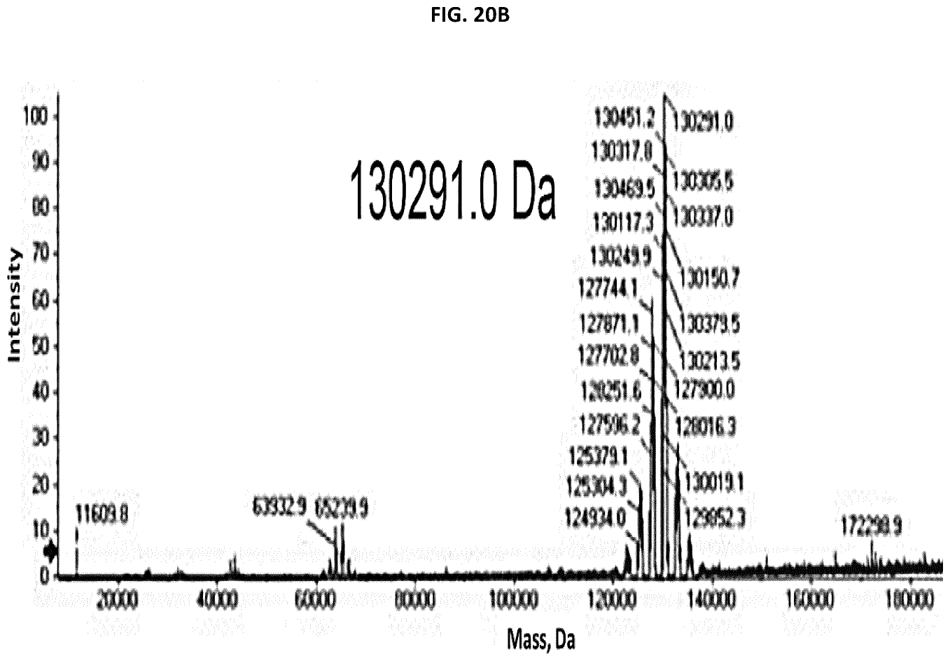

FIG. 20: MS analysis of intact BEAT (A) native, (B) enriched.

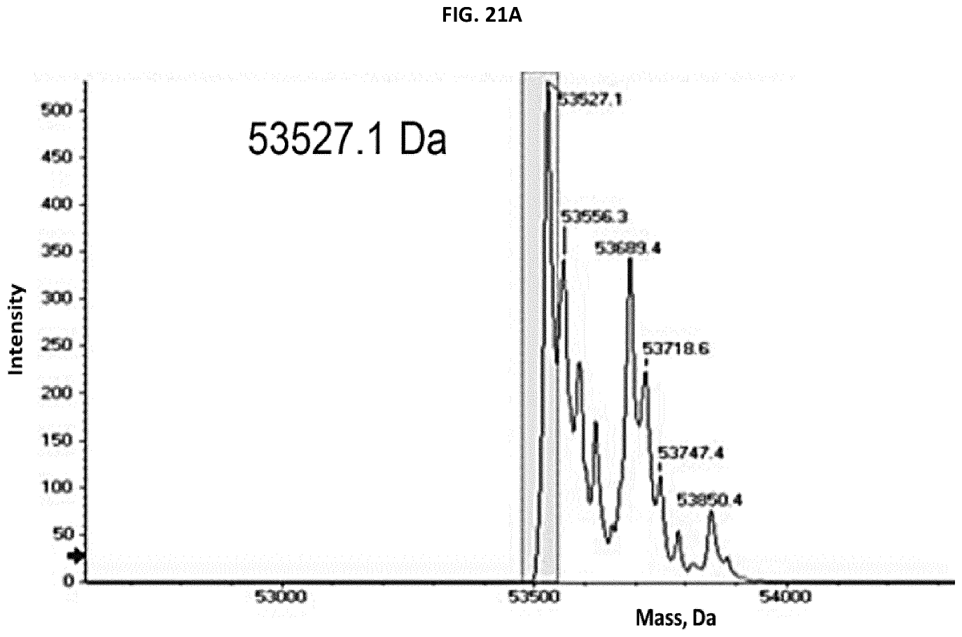

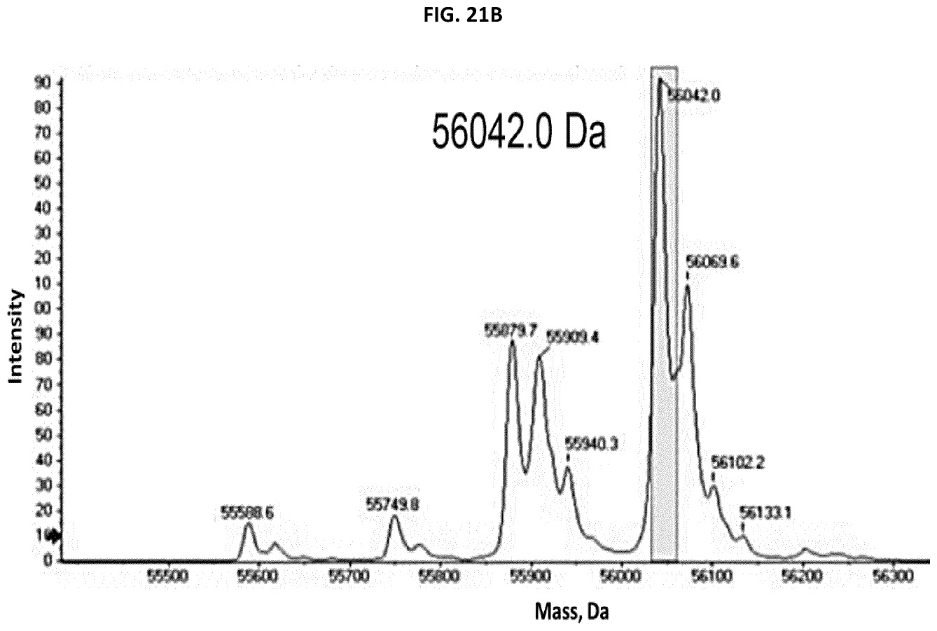

FIG. 21: MS analysis of reduced BEAT—ScFv-Fc, native (A), enriched (B).

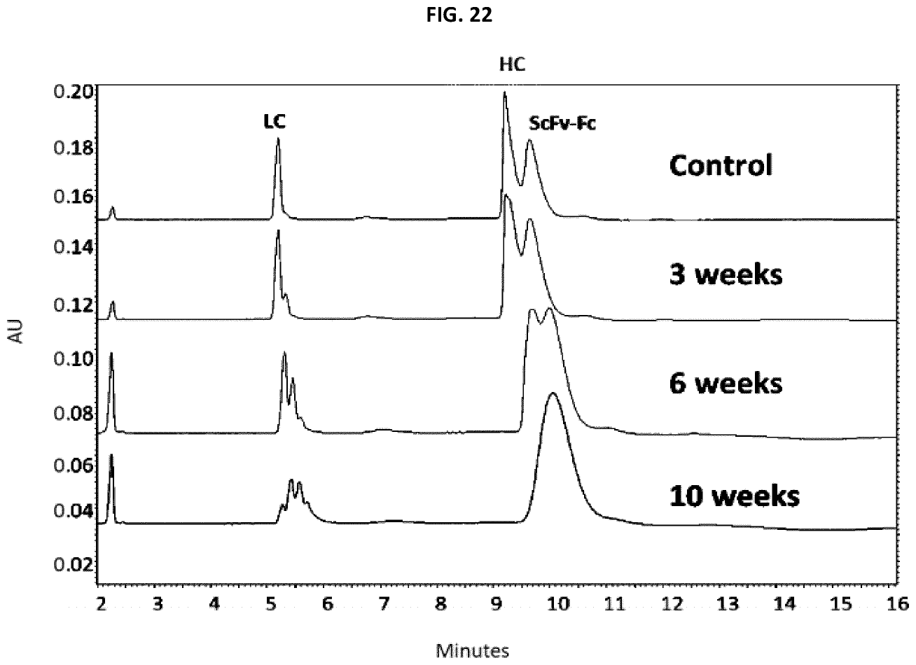

FIG. 22: Impact of glycation of lyophilized BEAT_Ab1 on HC and ScFv, 3 months time point, analysis on reduced CE-SDS.

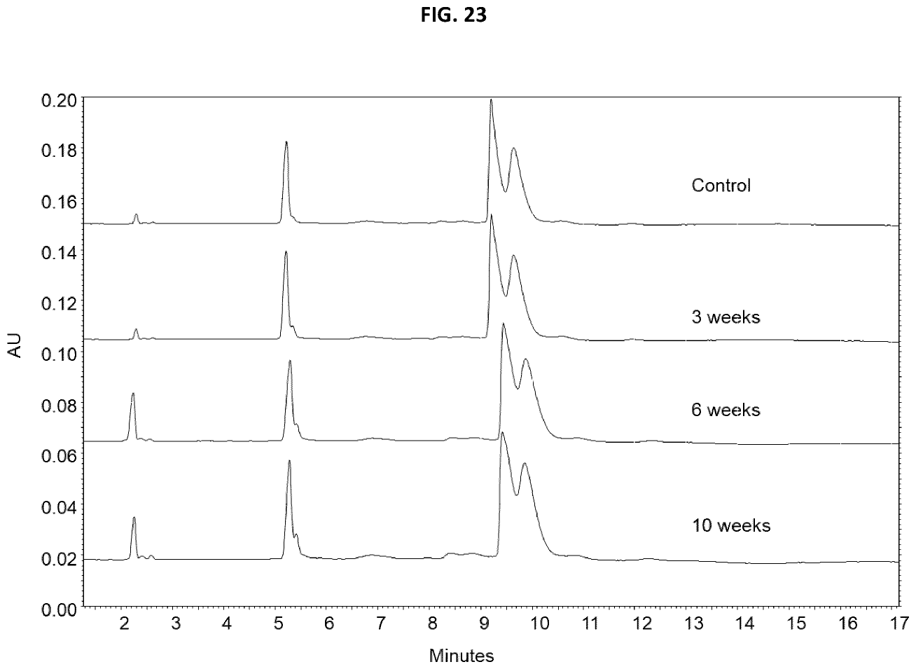

FIG. 23: Impact of glycation of BEAT_Ab1 in liquid on HC and ScFv, 4 months time point, analysis on reduced CE-SDS.

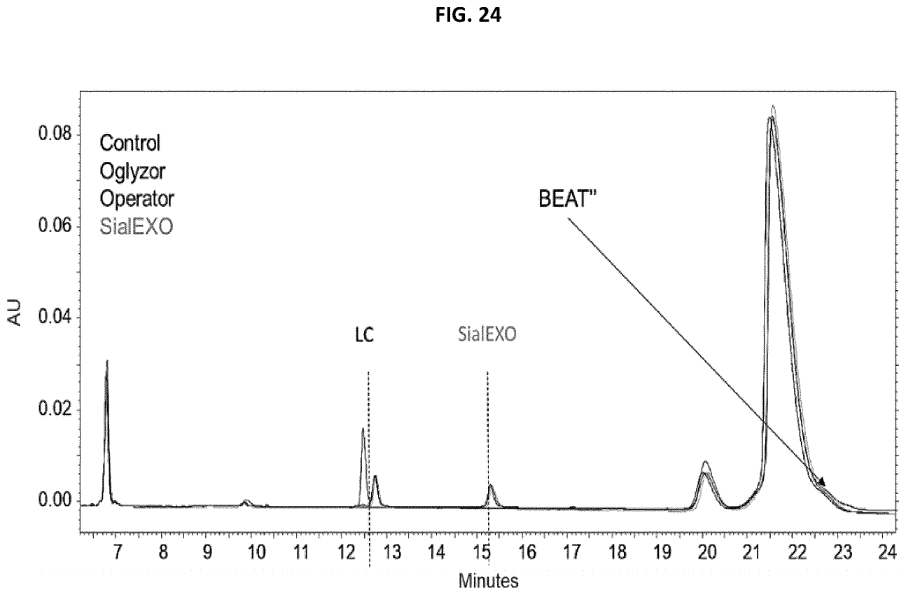

FIG. 24: Non-reduced CE-SDS profiles obtained for BEAT_Ab1_BDS after OpeRATOR, OglyZOR and SialEXO treatment in native conditions.

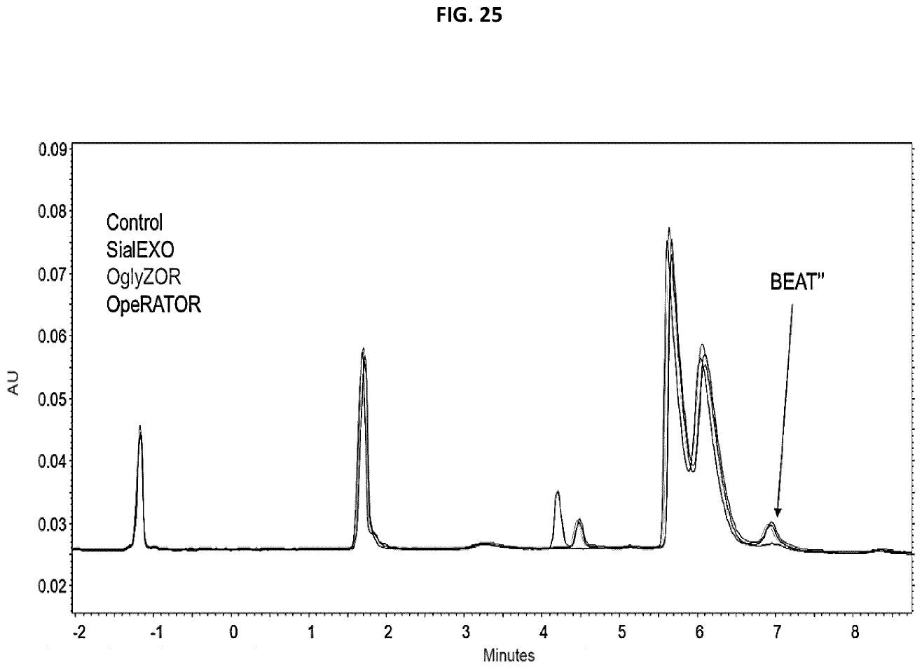

FIG. 25: Reduced CE-SDS profiles obtained for BEAT_Ab1_BDS after OpeRATOR, OglyZOR and SialEXO treatment in native conditions.

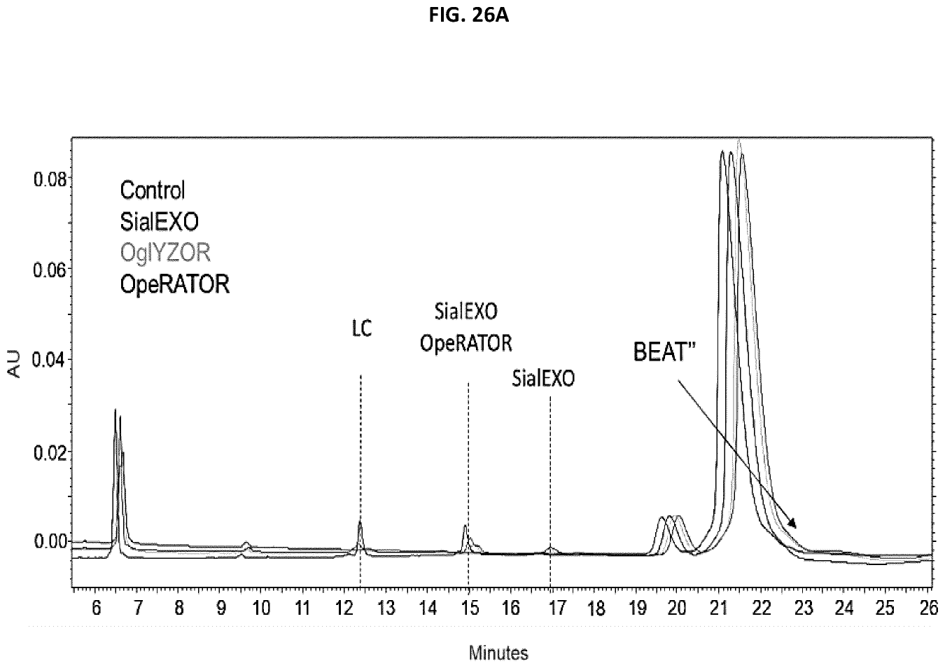

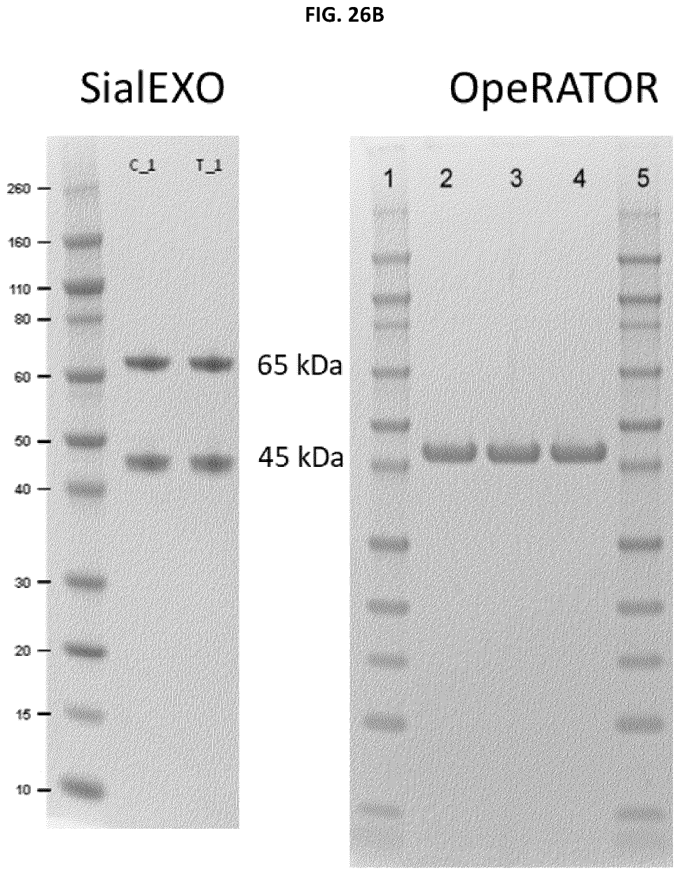

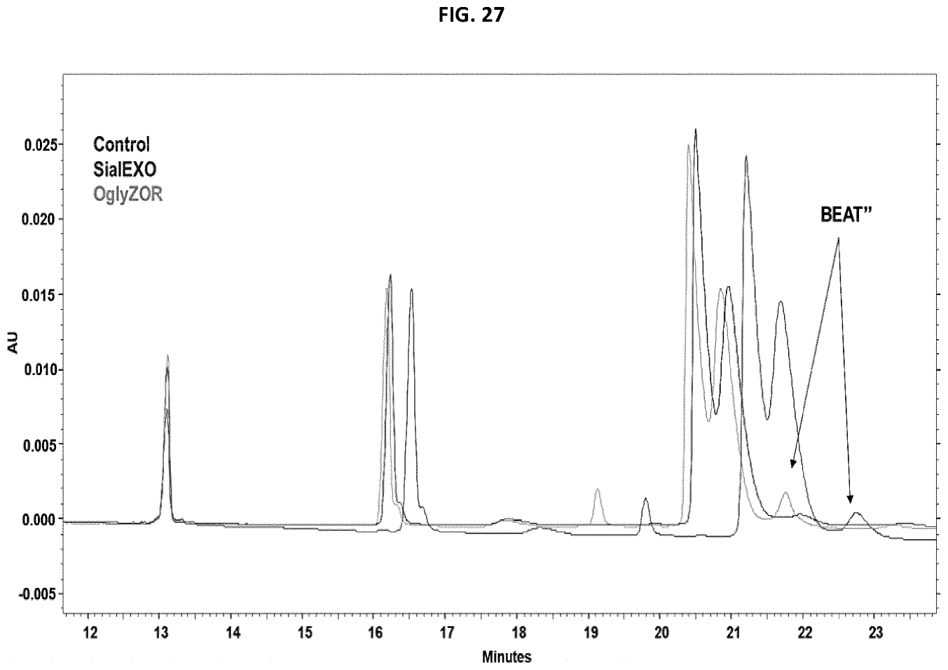

FIG. 26: (A) Non-reduced CE-SDS profiles obtained for BEAT_Ab1_BDS after OpeRATOR, SialEXO and OglyZOR treatment under denaturing condition; (B) SDS-PAGE gel for SialEXO and OpeRATOR.

FIG. 27: Reduced CE-SDS SDS profiles obtained for BEAT_Ab1_BDS after SialEXO and OglyZOR treatment under denaturing condition.

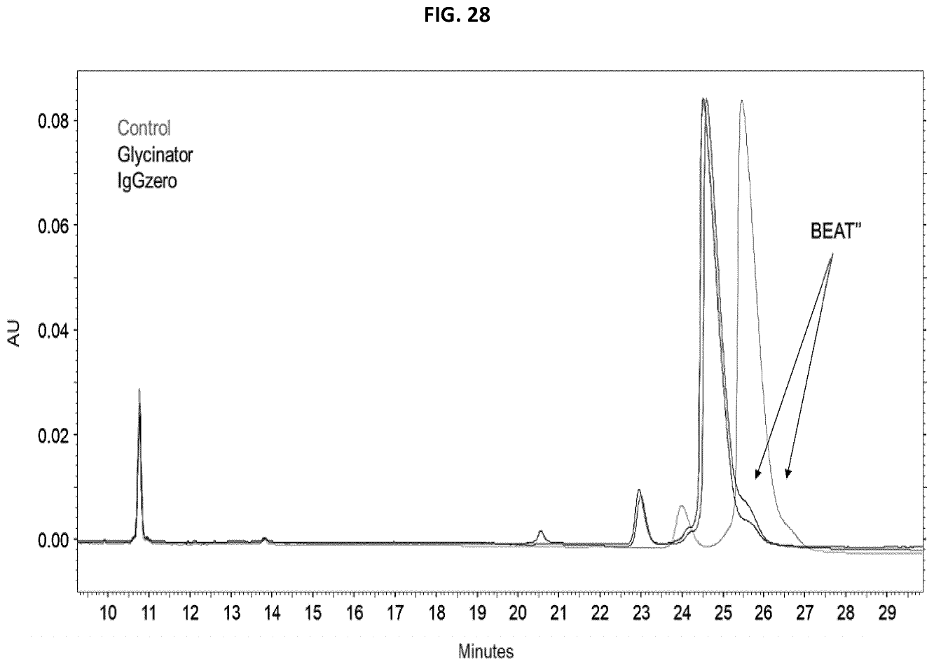

FIG. 28: Non-reduced CE-SDS profiles obtained for BEAT_Ab1_BDS after GlyciNATOR and IgGZERO treatment, under native conditions.

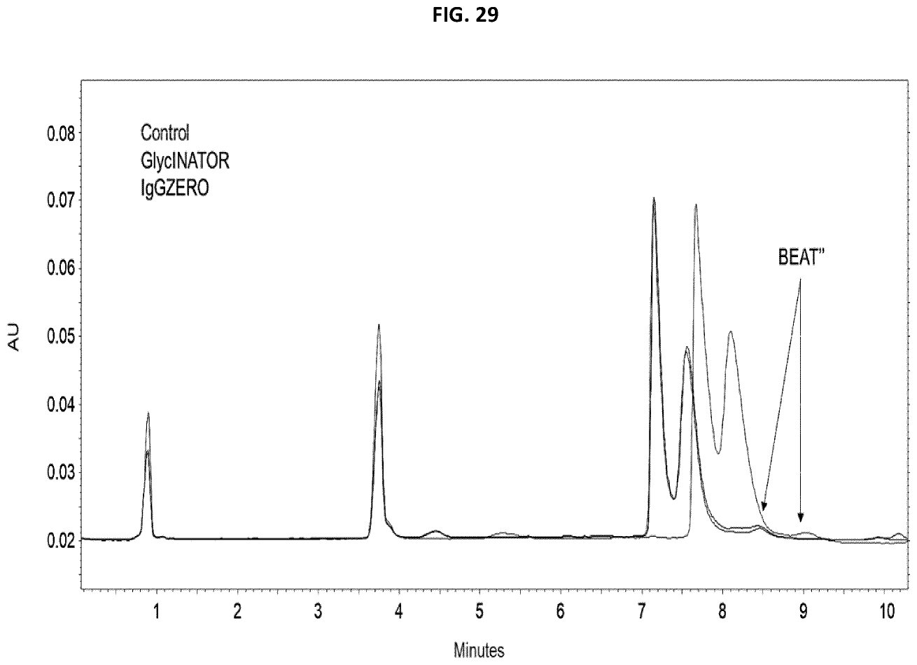

FIG. 29: Reduced CE-SDS profiles profiles obtained for BEAT_Ab1_BDS after GlyciNATOR and IgGZERO treatment, under native conditions.

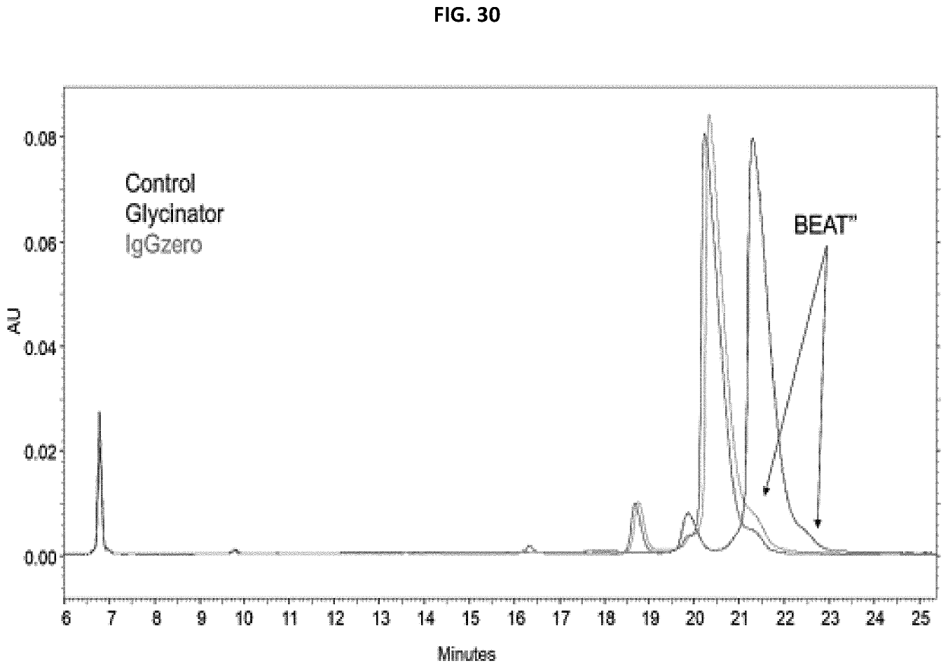

FIG. 30: Non-reduced CE-SDS profiles obtained for BEAT_Ab1_BDS after GlyciNATOR and IgGzero treatment under denaturing condition.

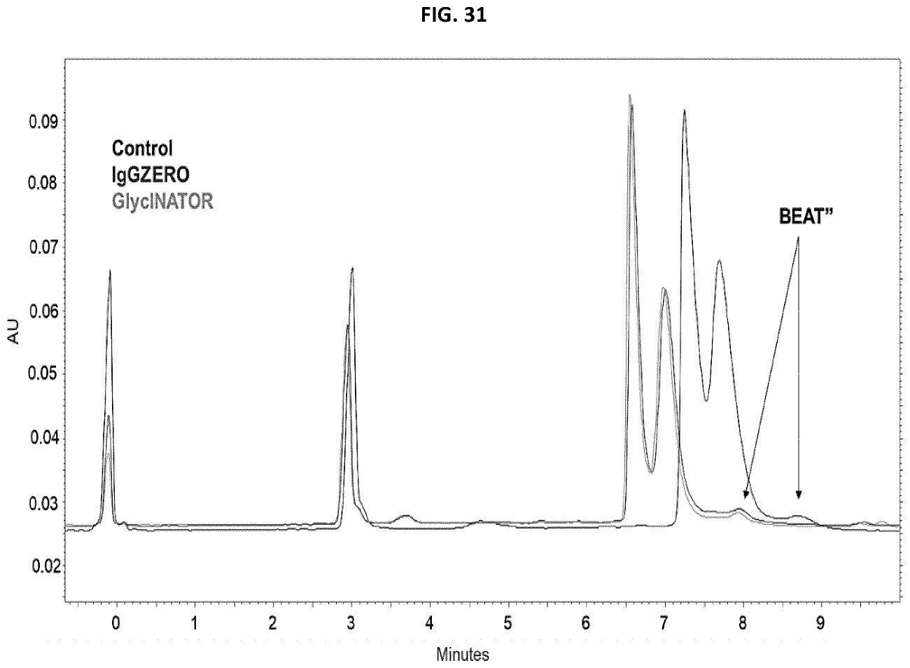

FIG. 31: Reduced CE-SDS profiles obtained for BEAT_Ab1_BDS after GlyciNATOR and IgGzero treatment under denaturing condition.

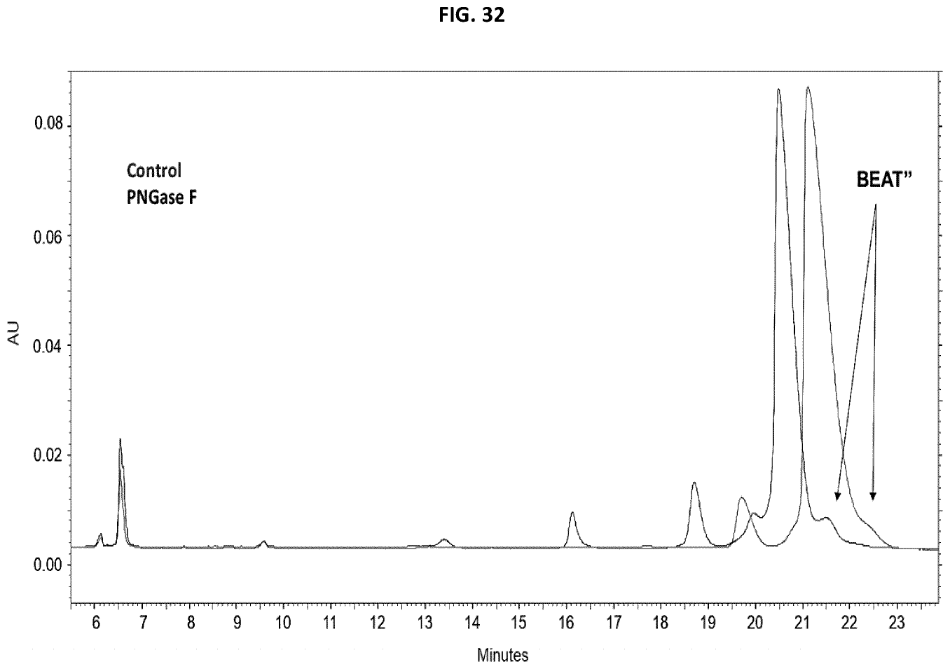

FIG. 32: Non-reduced CE-SDS profiles obtained for BEAT_Ab1_BDS after PNGase F treatment in native conditions.

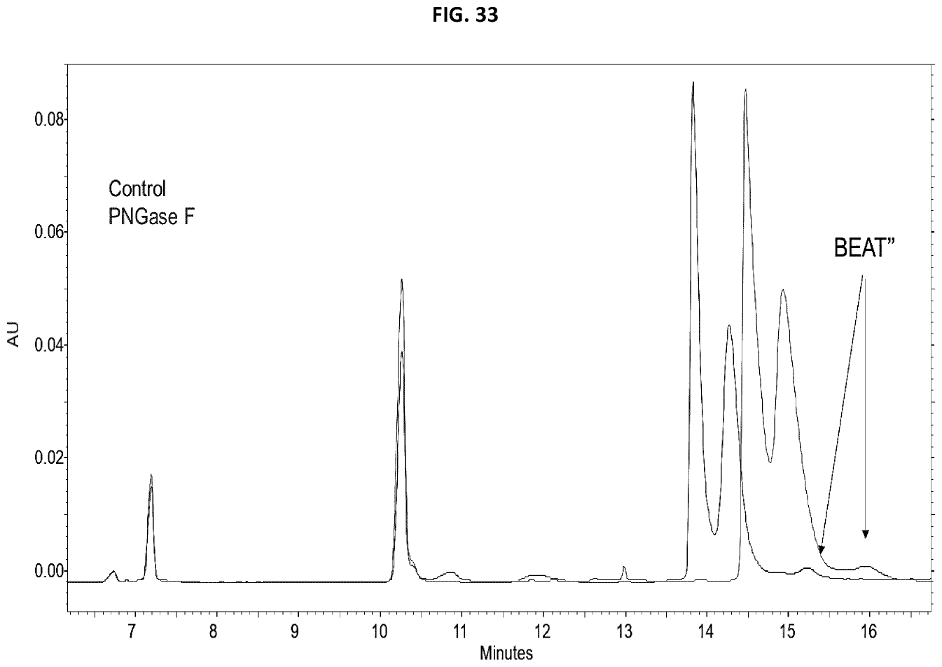

FIG. 33: Reduced CE-SDS profiles obtained for BEAT_Ab1_BDS after PNGase F treatment in native conditions.

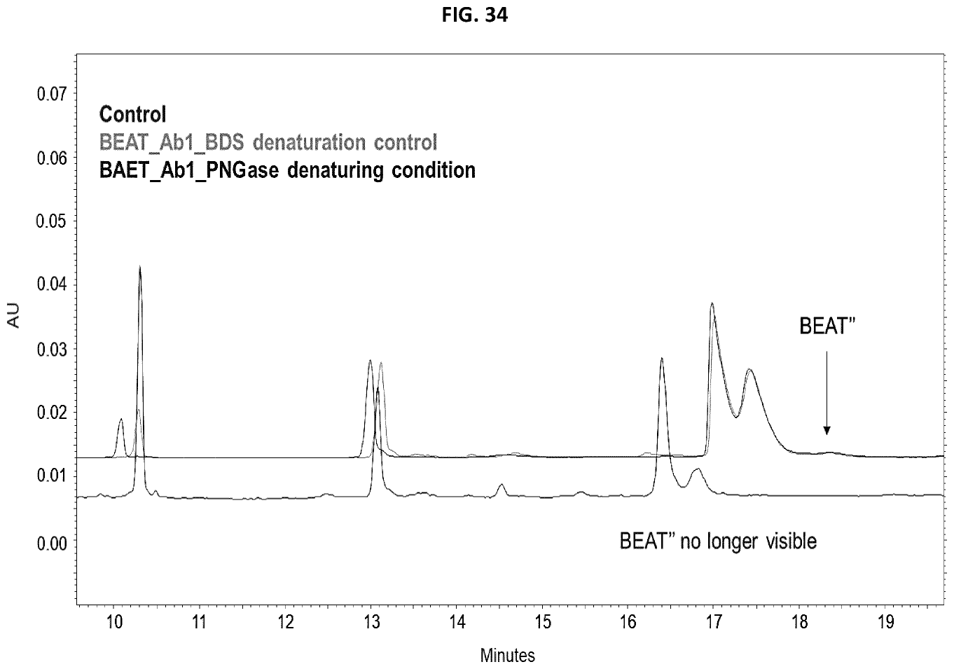

FIG. 34: Reduced CE-SDS profiles obtained for BEAT_Ab1_BDS after PNGase F treatment under denaturing condition (New England Biolabs protocol).

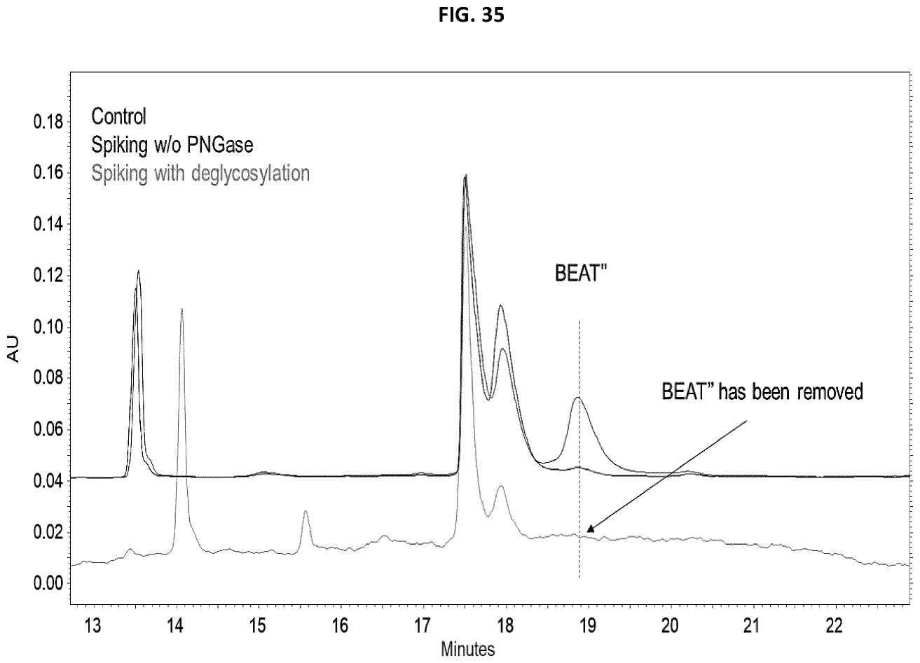

FIG. 35: Reduced CE-SDS profiles obtained for BEAT_Ab1_BDS spiked with 80% BEAT″ enriched material with and without PNGase F under denaturing condition treatment and control condition.

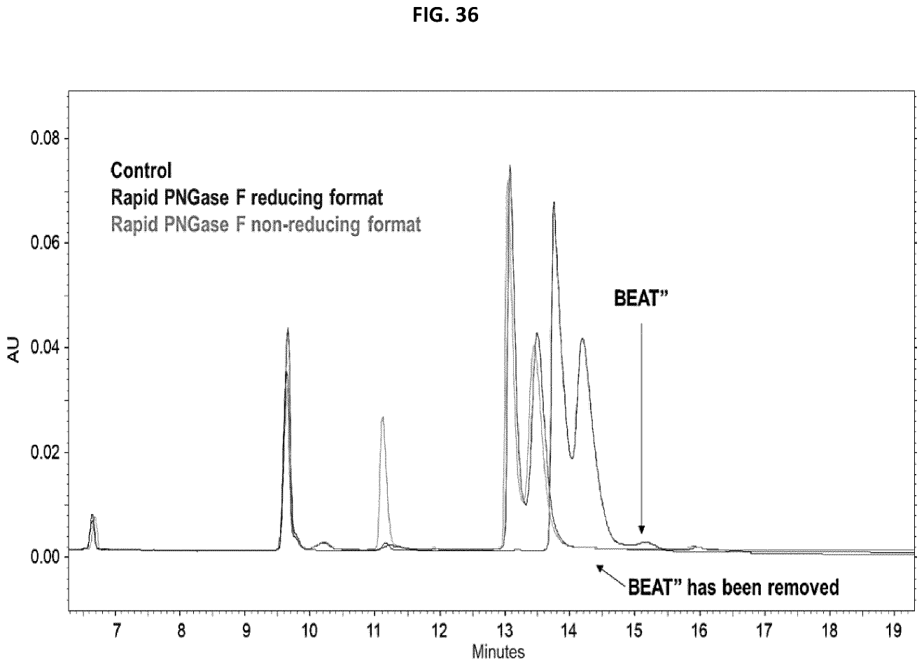

FIG. 36: Reduced CE-SDS profiles obtained for BEAT_Ab1_BDS after rapid PNGase F reducing and non-reducing format treatment.

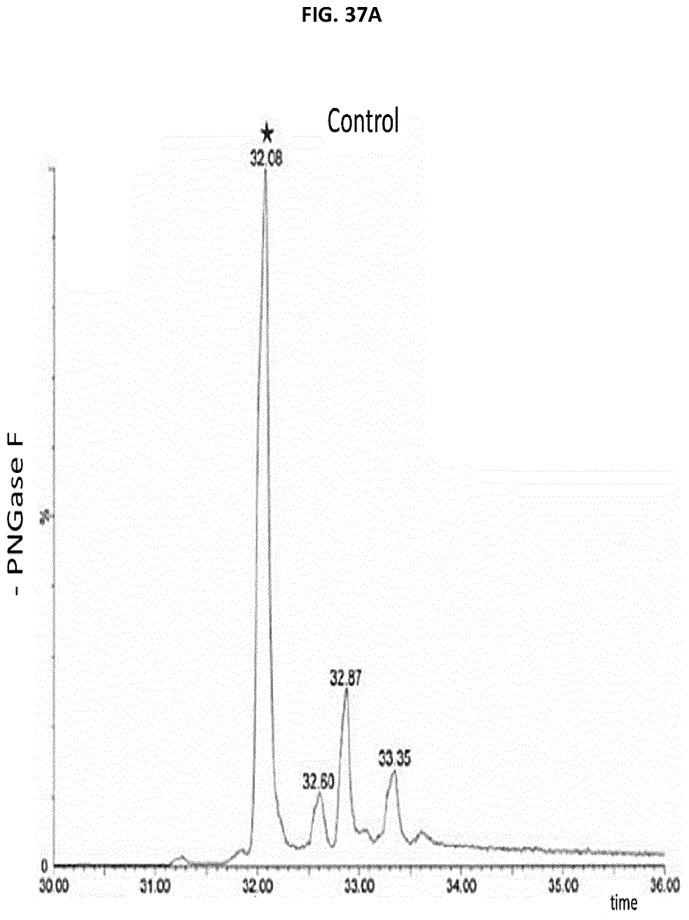

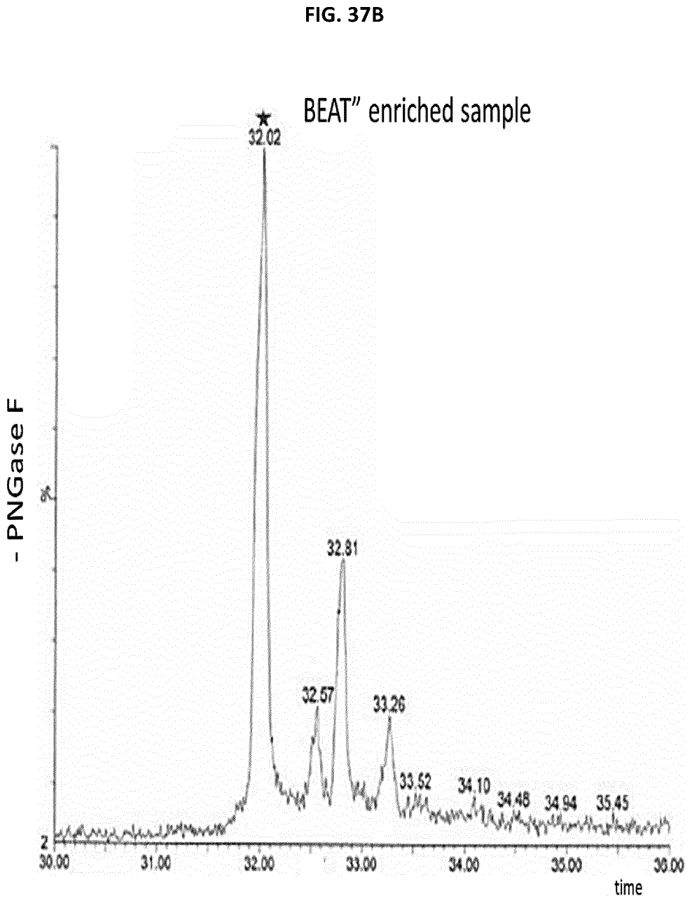

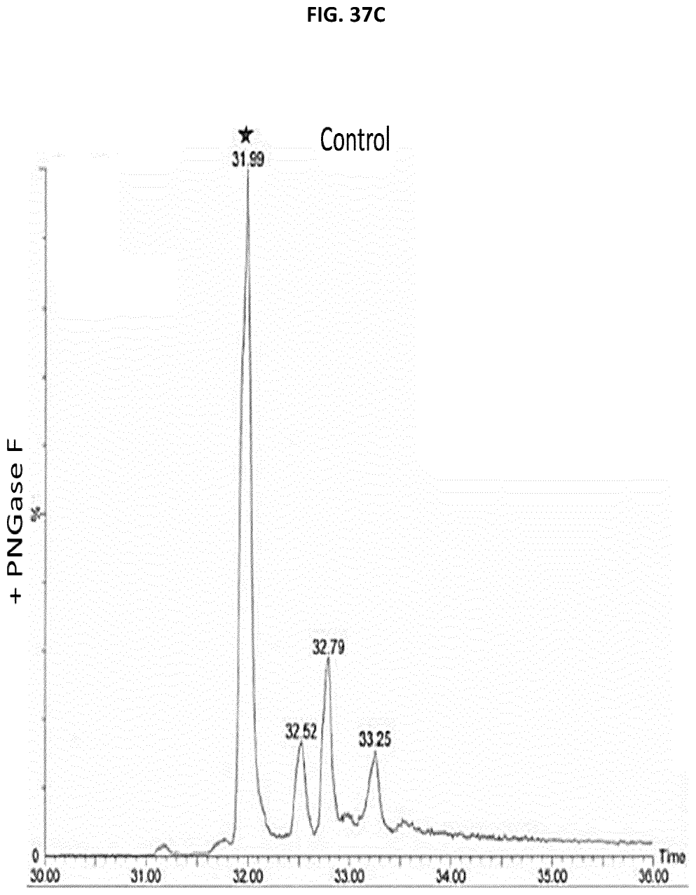

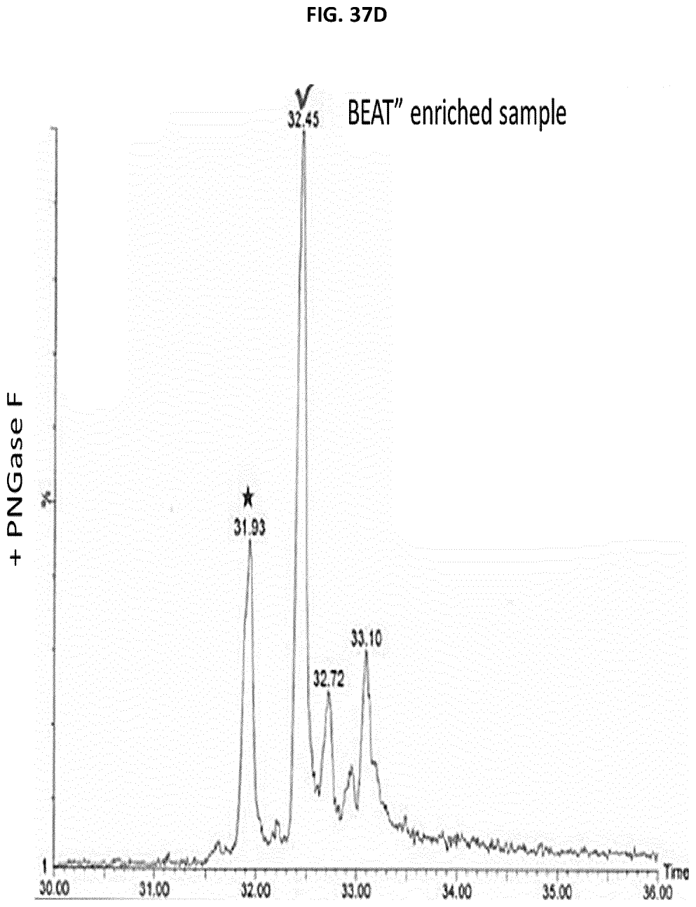

FIG. 37: UPLC-UV-MS^{E} analysis of Trypsin/Lys-C digested samples with or without PNGase F treatment showing Extracted Ion Chromatograms (EICs) encompassing native and deamidated scFv-Fc peptide 101-158 (charge state: 4+). Star marks indicate native scFv-Fc 101-158. Tick marks indicate PNGase F-induced scFv-Fc peptide 101-158 deamidation.

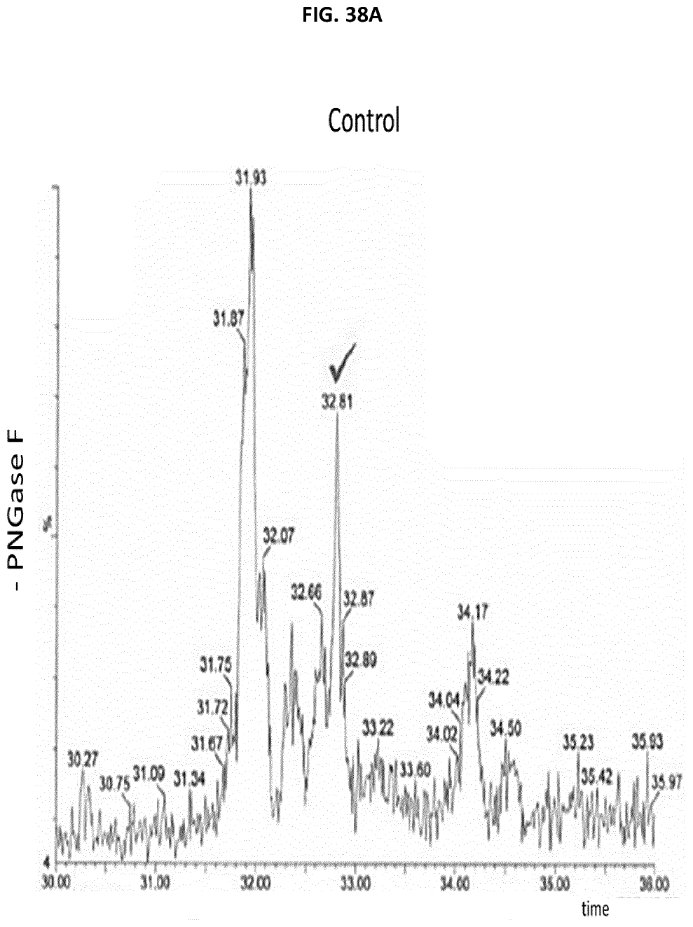

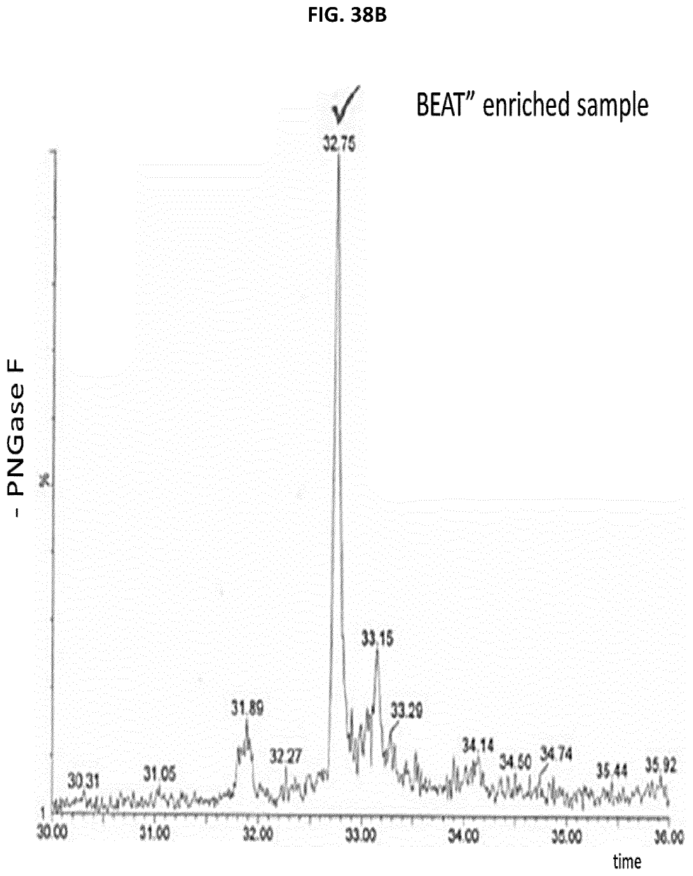

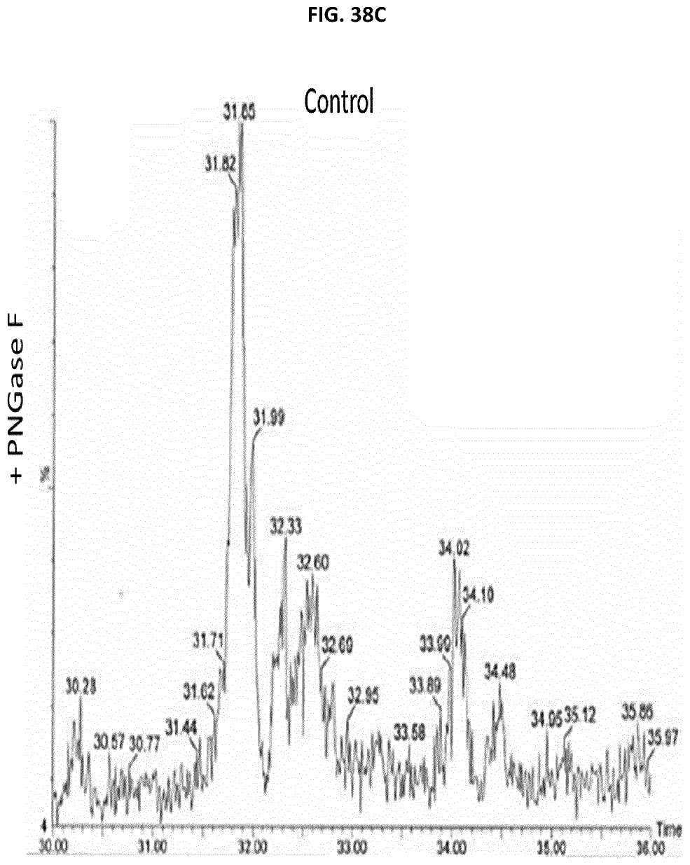

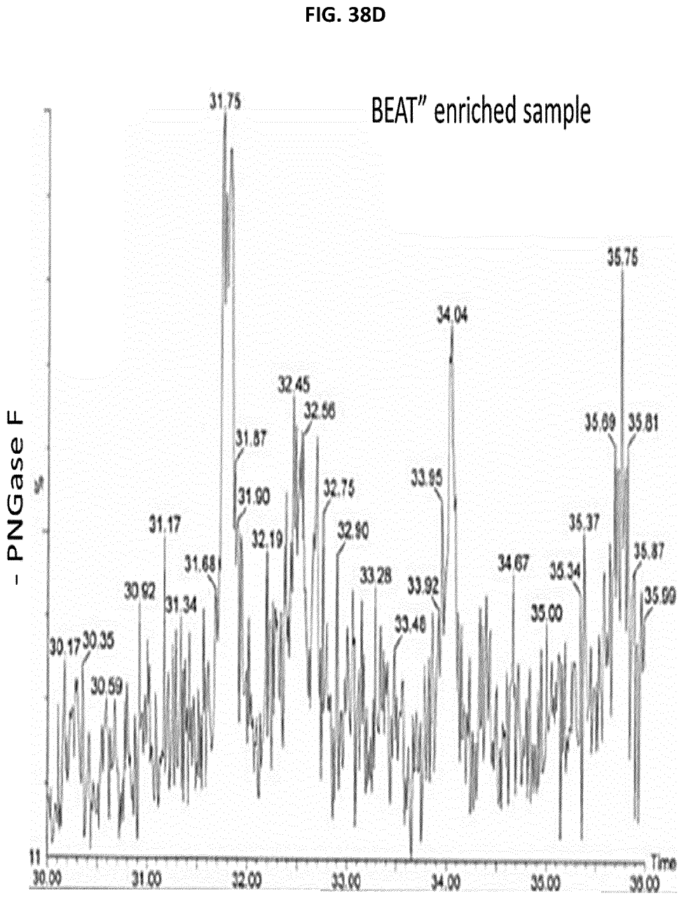

FIG. 38: UPLC-UV-MS^{E} analysis of Lys-C/Trypsin-digested samples with or without PNGase F treatment showing Extracted Ion Chromatograms (EICs) targeting glycosylated scFv-Fc peptide 101-158 (charge state: 5+). Tick marks indicate scFv-Fc peptide 101-158 substituted by G2FS2.

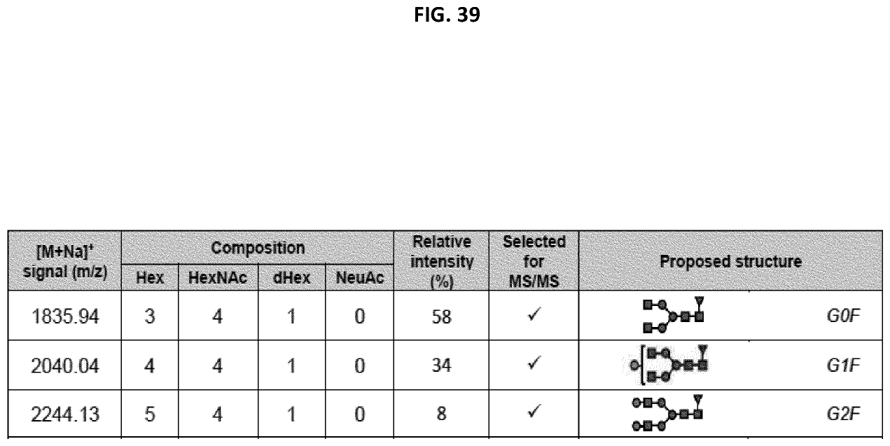

FIG. 39: Summary of the BEAT_Ab1 Fc N-glycans identified by MALDI-MS and MS/MS analyses.

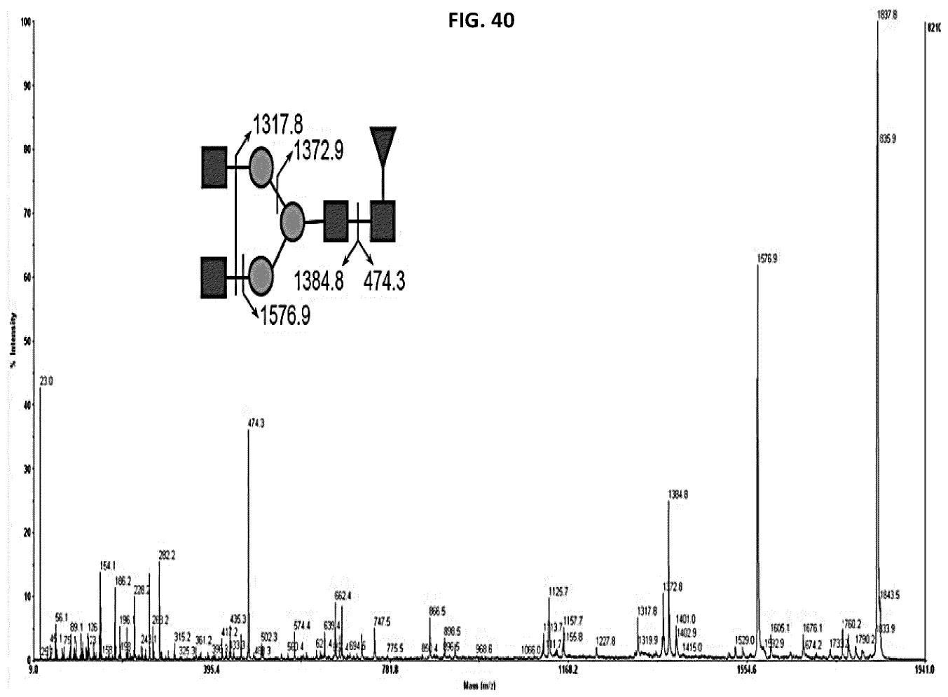

FIG. 40: MALDI-TOF-TOF mass spectrum obtained from permethylated Control BEAT_Ab1-BDS N-glycan at m/z 1835.

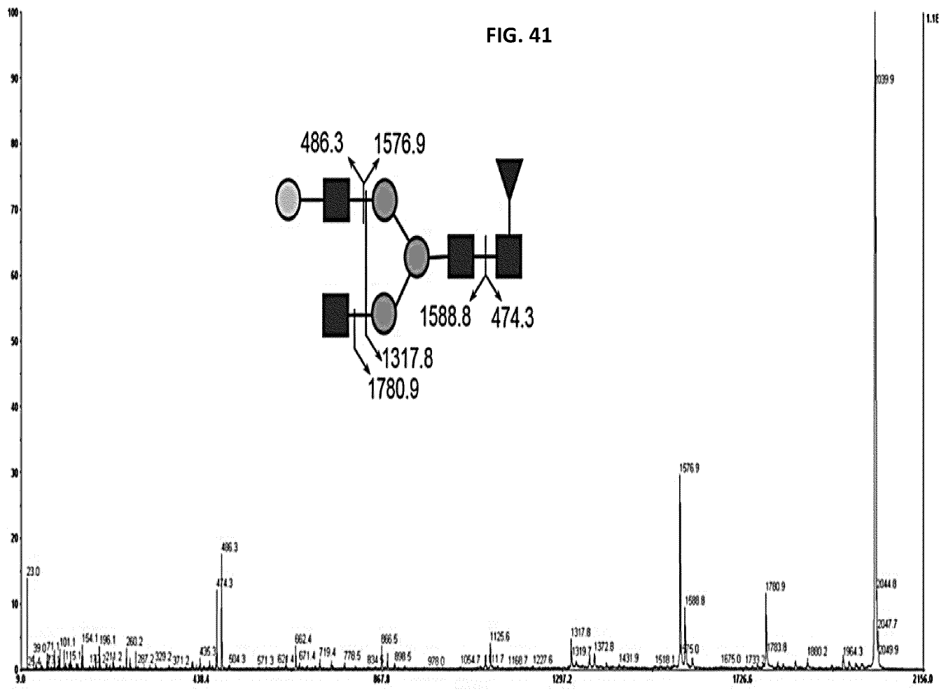

FIG. 41: MALDI-TOF-TOF mass spectrum obtained from permethylated Control BEAT_Ab1-BDS N-glycan at m/z 2040.

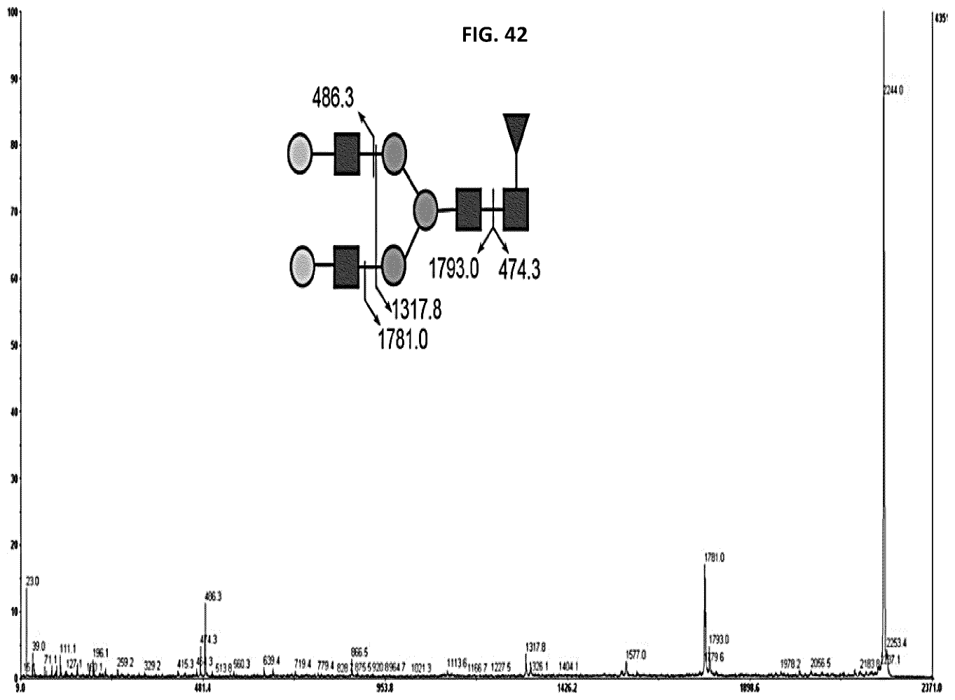

FIG. 42: MALDI-TOF-TOF mass spectrum obtained from permethylated Control BEAT_Ab1-BDS N-glycan at m/z 2244.

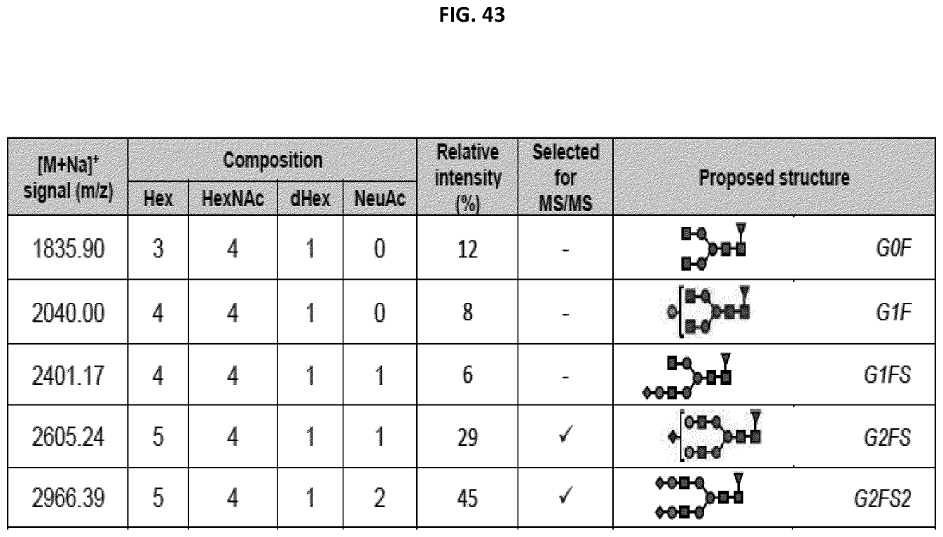

FIG. 43: Summary of the N-glycans present at non-consensus N-glycosylation site of EP180/BEAT″ enriched sample identified by MALDI-MS and MS/MS analyses.

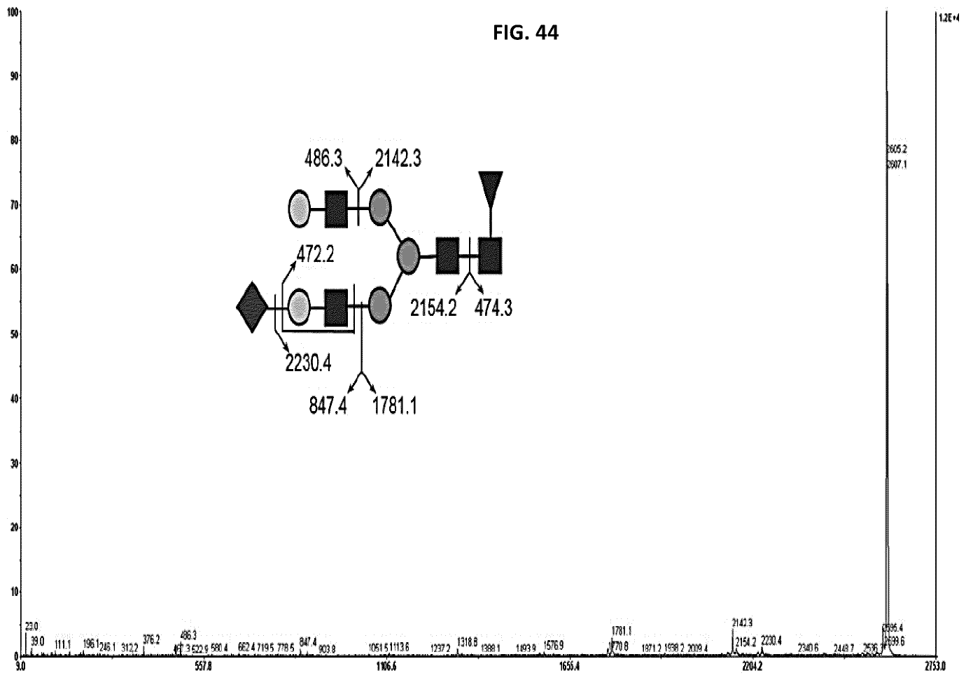

FIG. 44: MALDI-TOF-TOF mass spectrum obtained from permethylated of EP180/BEAT″ enriched sample N-glycan at m/z 2605.

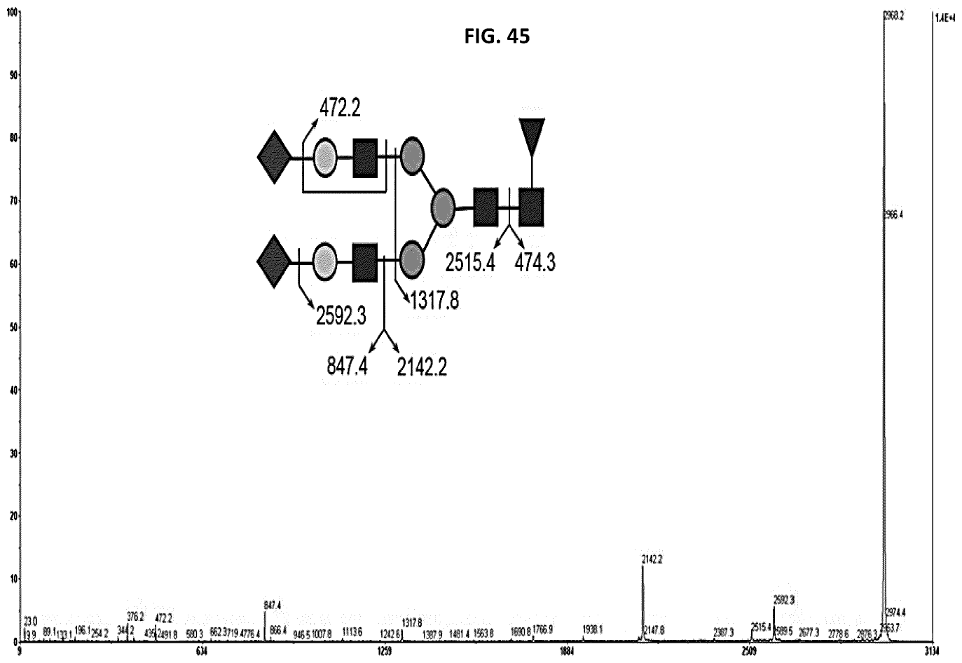

FIG. 45: MALDI-TOF-TOF mass spectrum obtained from permethylated of EP180/BEAT″ enriched sample N-glycan at m/z 2966.

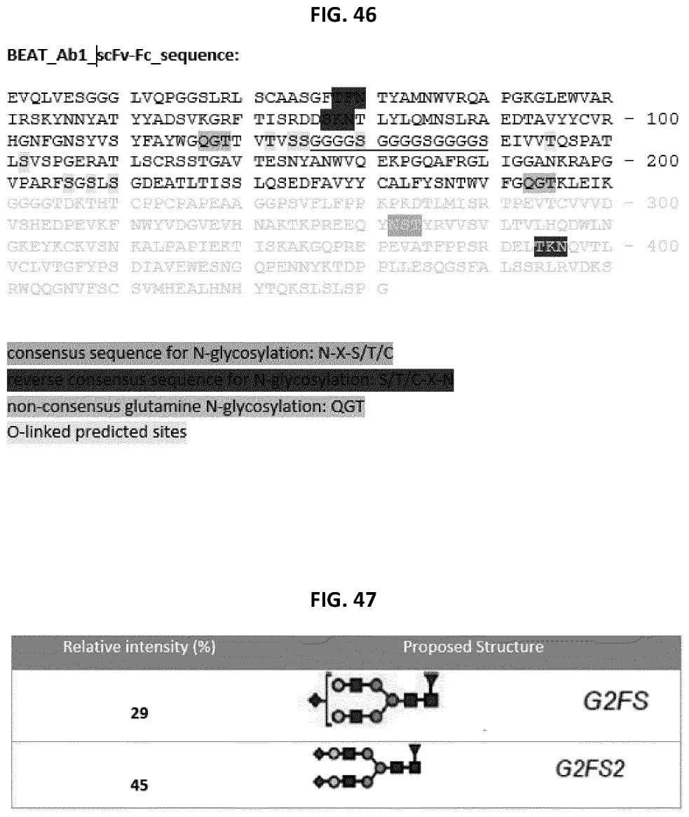

FIG. 46: Glycosylation sites of BEAT_Ab1 of the ScFv-Fc

FIG. 47: Two potential glycated structure for BEAT″

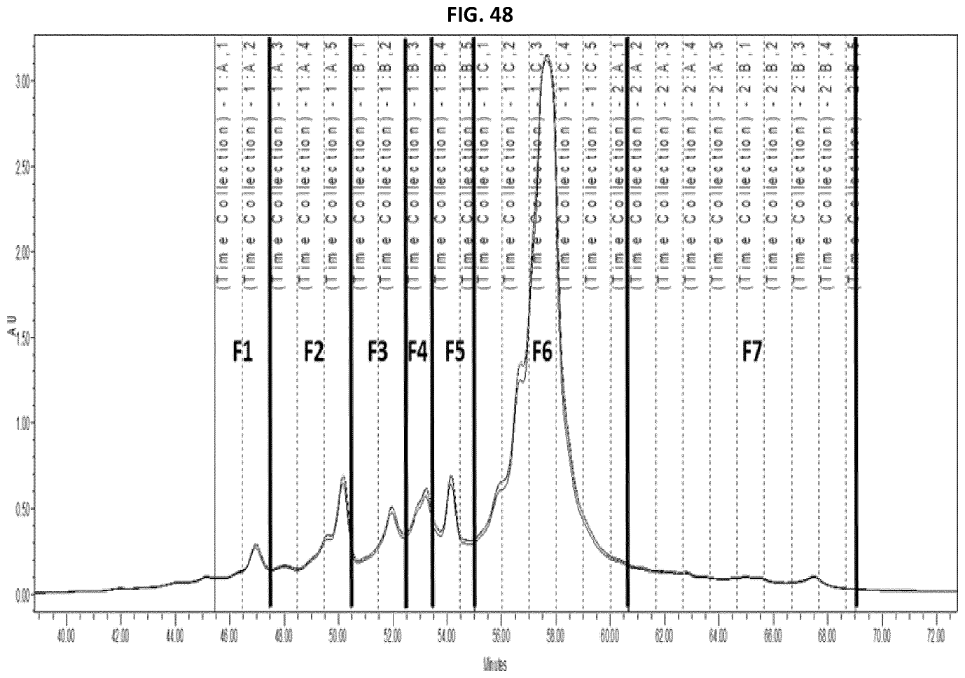

FIG. 48: BEAT_Ab1 CEX profile

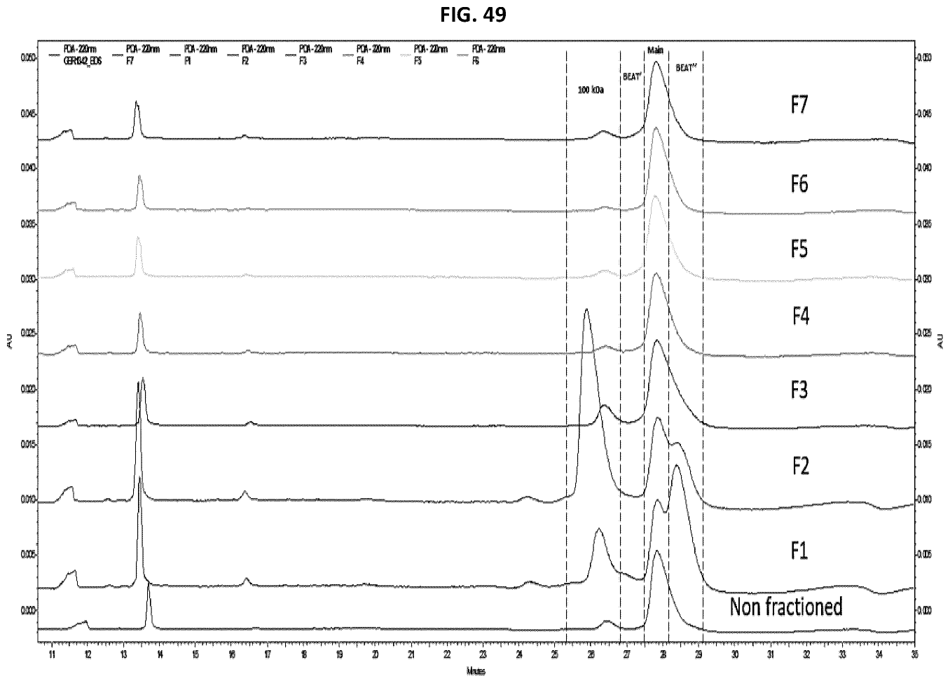

FIG. 49: BEAT_Ab1 CEX fractions overlay after non-reduced CE-SDS

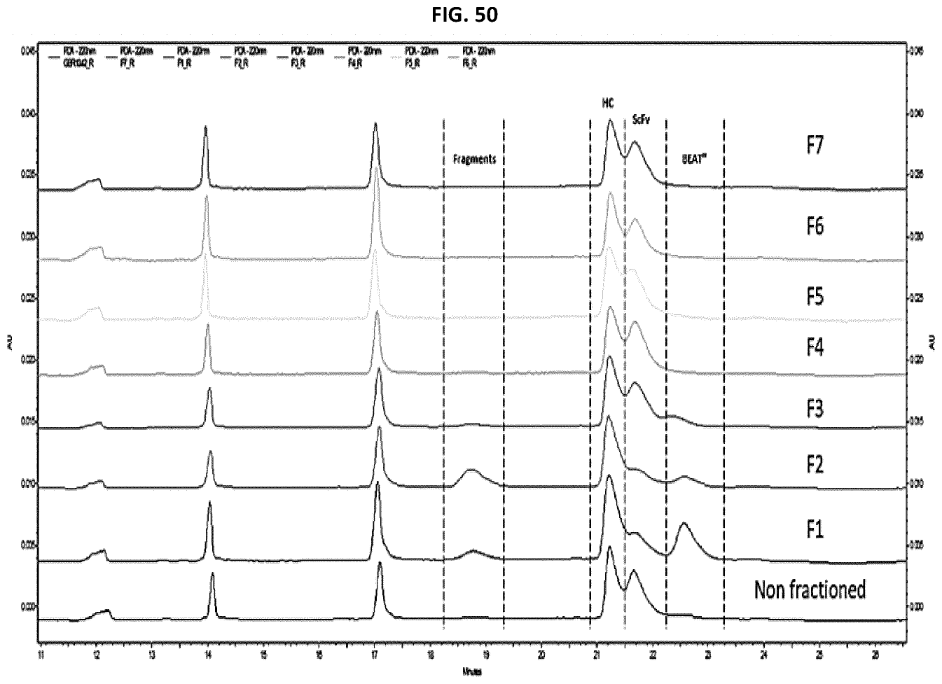

FIG. 50: BEAT_Ab1 CEX fractions overlay after reduced CE-SDS

### EXAMPLE 1: BEAT_AB1 CE-SDS PROFILE

BEAT_Ab1 was expressed by CHO-S cells cultured for around 14 days of culture according to the manufacturer's instructions. BEAT_Ab1 was next purified by a purification process including steps of affinity chromatography and ion exchange chromatography. The bulk drug substance obtained after the purification steps was analyzed by non-reduced and reduced capillary electrophoresis-sodium dodecyl sulfate polymer-filled capillary gel electrophoresis (CE-SDS) to assess its purity.

CE-SDS analysis was performed according to manufacturer instructions. In short 1 mg/mL of each BEAT_Ab1 sample in sample buffer containing 2 μL of Internal Standard+5 μL of 2-ME (for reducing condition) or 5 μL of Iodoacetamide (IAM) (for non-reducing condition) with a final volume of 100 μL was heated at 70° C. for 10 min (for reducing condition) or at 50° C. for 5 min (for non-reducing condition), and then the solution mixture was analyzed with Beckman PA800 CE system equipped with UV diode-array detector (220 nm wavelength) and a bare-fused silica capillary with LD=20 cm, LT=30.2 cm, and inner diameter of 50 μm, using provided instrument run conditions.

The non-reduced CE-SDS profile (FIG. 1) shows a main monomer peak (BEAT), its variants (BEAT′, BEAT″) and fragments (100 kDa, 75 kDa, LC). The reduced CE-SDS profile (FIG. 2) shows three main peaks: BEAT_Ab1 light Chain (LC), Heavy Chain (HC), and ScFv-Fc, as well as reduced fragments and variants. In non-reduced capillary gel electrophoresis, an unexpected peak have been found (BEAT″). This peak is present after the main peak meaning that this species is heavier than BEAT_Ab1 antibody.

In order to characterize BEAT″ species, different experiments have been made to investigate the origin of BEAT″ CE-SDS peak.

### EXAMPLE 2: INVESTIGATION OF THE BEAT″ CE-SDS PEAK IDENTITY

### CE-SDS Artefact

First we investigated whether BEAT″ was an artifact generated during the CE-SDS analysis. In particular we focused on non-reduced CE-SDS analysis and we tested different time and temperature conditions for the sample preparation, according to Table 1.

TABLE 1
Time and temperature
conditions used for
denaturation

| Time | |
|---|---|
| (min) | Temperature (° C.) |
| 5 | 50 |
| 5 | 70 |
| 5 | 90 |
| 10 | 50 |
| 10 | 70 |
| 10 | 90 |
| 15 | 50 |
| 15 | 70 |
| 15 | 90 |

The results reported in FIG. 3 show that BEAT″ peak is not affected by the heating time and temperature conditions. Therefore, BEAT″ species is not generated by the sample preparation for CE-SDS analysis.

### Signal Peptide

Next we investigated whether BEAT″ peak was related to the non-removal of the signal peptide on BEAT_Ab1. In fact, a failed removal of BEAT_Ab1 signal peptide would lead to a protein with higher mass, justifying the CE-SDS results shown in Example 1.

For this experiment, the samples were separated by 1D SDS-PAGE and subsequently stained by colloidal Coomassie Brilliant Blue (Sigma Aldrich) and Silver, using standard protocols. The gel images were digitized using a flatbed scanner with 300 dpi resolution. The identification of the protein bands was carried out by (Liquid Chromatography-Electrospray Ionisation—Mass Spectrometry) LC-ESI-MS and MS/MS measurement after enzymatic protein digestion. In particular, LC-ESI-MS and -MS/MS mass spectra were obtained using the UltiMate® 3000 RSLCnano System (Thermo Fisher Scientific) coupled to a Q Exactive Orbitrap mass spectrometer (Thermo Fisher Scientific). The separation of the peptides was performed with reversed-phase (RP) chromatography. Separator column Acclaim PepMap RSLC C18 column (300 μm I.D.×150 mm, 2 μm particle size, 100 Å pore size, Thermo Fisher Scientific) was used. Eluents were A: water/0.1% formic acid; B: acetonitrile/0.1% formic acid. The peptides were separated using a segmented gradient from 2% B to 50% B in 32 min at 40° C. with a flow rate of 5 μL/min. MS and MS/MS spectra (produced with Higher Energy Collisional Dissociation, HCD) were recorded in positive ion mode with internal mass calibration. Blank measurements with injection of 0.1% TFA were acquired before each gel band sample to evaluate background signals (carry over). The MS data sets were analyzed by the ProteinScape 2 bioinformatics platform (Bruker Daltonics, Protagen AG). Protein identification was achieved by database searching. Hereby the fragment mass spectra were matched against an in-house database, consisting of the NCBI human and rodent protein database (http://www.ncbi.nlm.nih.gov/) and the manually inserted protein sequence.

The following protein modifications have been measured:
Propionamide (Cys) and Cys, Dehydroalanine formation, introduced by SDS-PAGE
Deamidation (Asn)
Oxidation (Met)
Pyro-glutamate formation (Gln and Glu at peptide N-terminus)
Acetylation (protein N-terminus)

All the bands selected for LC-ESI-MS analysis are visible in FIG. 4 and the Total Ion Current (TIC) profiles for the band of interest (those containing BEAT″—Band 1 and 4) are presented in FIG. 5. In all analyzed bands BEAT_Ab1 was identified. Peptides matching the signal peptide of BEAT_Ab1 or part of it were not identified, indicating that BEAT″ is not related to the failed removal of the signal peptide.

### Host Cell Proteins

As BEAT″ peak was shown not to be related to the missed removal of the signal peptide, we proceeded by investigated whether it was related to the presence of Host Cell Proteins (HCPs).

HCP analysis and signal peptide analysis have been performed within the same experiment with 1D gel. The selected band was run through mass spectrometer and the results were compared to existing database to see if HCP could be detected. HCPs analysis could not be performed on band 3, because of a contamination. However this band did not correspond to the one containing BEAT″ so this did not have an impact on the outcome of this analysis. For all the analyzed bands, no HCPs have been detected by the reference database used. This experiment therefore demonstrated that BEAT″ signal is not related to HCPs.

### EXAMPLE 3: BEAT″ ENRICHMENT AND ACTIVITY EXPERIMENTS

Given that from the previous experiments it was not possible to draw a final conclusion on the origin of BEAT″ CE-SDS peak, we tried to separate BEAT and BEAT″ species by size exclusion chromatography (SE-HPLC). It was not possible to separate these species by analytical SEC (see FIG. 6), because of the lower resolution power comparing to CE-SDS. To overcome this limit we developed a method, based on SE-HPLC, to generate highly enriched BEAT″ material, to use for affinity and potency studies.

### Enrichment Experiment

First we tested and adapted the volume of material to inject on the SE-HPLC column in order to maximize the volume to inject without affecting the behavior of our antibody inside the column. To do so, four different injection volumes of BEAT_Ab1_BDS were tested, as indicated in Table 2.

TABLE 2
Correspondence between volume and
quantity injected on the column

| | | | | |
|---|---|---|---|---|
| Volume injected (μL) | 50 | 150 | 200 | 250 |
| Quantity injected (μg) | 285 | 855 | 1140 | 1425 |

As shown in FIG. 7, when volumes above 150 μL (855 μg) are injected, the profile obtained is not the one usually expected, probably due to the column overload. Therefore a volume of 150 μL was used for the SE-HPLC column injection.

To generate a BEAT″ enriched material by SE-HPLC, the main peak was split into 15 fractions (representing approximately between 2% and 5% of the main peak area each), as shown in FIG. 8. Of these 15 fraction, 12 have been analyzed by non-reduced CE-SDS to measure which fraction gives the best enrichment level (the amount of the remaining 3 fractions was insufficient for analysis).

TABLE 3
Relative peak area obtained after non-reduced CE-SDS for the 13 collected fractions and BEAT_Ab1 BDS

| Peak | | | | | | | | | | | | | |
|---|---|---|---|---|---|---|---|---|---|---|---|---|---|
| Name | BEAT_Ab1_BDS | F1 | F2 | F3 | F4 | F5 | F8 | F10 | F11 | F12 | F13 | F14 | F15 |
| LC | 0.6 | 0.7 | 0.5 | 0.3 | 0.4 | 0.5 | 0.6 | 0.7 | 0.6 | 0.4 | 0.4 | 0.6 | 0.7 |
| Fragment | 0.2 | 0.0 | 0.0 | 0.0 | 0.0 | 0.0 | 0.0 | 0.0 | 0.0 | 0.0 | 0.0 | 0.0 | 0.2 |
| 75 kDa | | | | | | | | | | | | | |
| Fragment | 0.3 | 0.0 | 0.0 | 0.0 | 0.0 | 0.0 | 0.0 | 0.0 | 0.0 | 0.0 | 0.0 | 0.0 | 3.3 |
| 80 kDa | | | | | | | | | | | | | |
| Fragment | 6.1 | 2.0 | 1.6 | 1.4 | 1.6 | 1.8 | 1.9 | 2.4 | 3.1 | 4.4 | 7.7 | 13.2 | 36.2 |
| 100 kDa | | | | | | | | | | | | | |
| BEAT′ | 1.6 | 4.9 | 2.3 | 1.9 | 1.9 | 1.6 | 1.7 | 1.5 | 1.7 | 1.4 | 0.0 | 0.0 | 0.0 |
| BEAT | 87.3 | 48.9 | 85.4 | 91.5 | 93.5 | 93.6 | 93.8 | 94.5 | 94.1 | 93.8 | 91.9 | 86.3 | 58.7 |
| BEAT″ | 3.1 | 42.4 | 9.8 | 4.9 | 2.6 | 2.6 | 1.9 | 1.0 | 0.6 | 0.0 | 0.0 | 0.0 | 0.0 |
| Aggregates | 0.9 | 1.2 | 0.5 | 0.0 | 0.0 | 0.0 | 0.0 | 0.0 | 0.0 | 0.0 | 0.0 | 0.0 | 0.0 |

As shown in Table 3, the fractions containing a high BEAT″ concentration are those at the beginning of the main peak (the first 5-10% of the main peak—F1 and F2).

In order to generate highly enriched material, fractions F1 to F3 (corresponding to the first 15% of the main peak) were collected and loaded on the SE-HPLC column. This process was repeated two to three times, depending on the level of enrichment needed, to collected material with higher enrichment level of BEAT″.

At the end of the enrichment cycles the material collected was pulled together, a buffer exchange was performed in a stable buffer and frozen at −80° C. With this process we were able to generate material enriched with BEAT″ up to the 80%, which was used for further activity experiments.

From the analysis of the highly enriched BEAT″ material we were able to see a correlation between non-reduced and reduced CE-SDS data, as shown in FIG. 9 and FIG. 10. The species identified as BEAT″ on non-reduced CE-SDS data (shoulder after the main peak) seems to correspond to the unknown peak after the ScFv-Fc peak on reduced CE-SDS data. Moreover we can observe a decrease of the ScFv-Fc part of the molecule correlated with an increase of the unknown peak for enriched material. Based on these data we hypothesized that the unknown peak corresponds to BEAT″ and that ScFv-Fc part of the molecule may carried the modification responsible of BEAT″. This hypothesis have been further verified using different techniques, including Biacore SPR analysis, cell based assay, affinity purification and statistical analysis by JMP software.

### Biacore Analysis

The purpose of this analysis is to measure the binding affinities of BEAT″ enriched material to the targets CD3ε 1-26-Fc and CD38 using Surface Plasmon Resonance (SPR). As shown in FIG. 11 the SE-HPLC fractions collected for Biacore analysis are: F1AA (BEAT″: 79.2%), F1AB (BEAT″: 58.2%), F1B (BEAT″: 23.8%), F3 (BEAT″: 4.2%). These fractions have been collected during the third cycle of enrichment and analyzed by CE-SDS to measure the level of enrichment.

Biacore measurements, shown in FIG. 12, indicate that all the tested fractions have decreased binding on both epitopes. This result could be explained by the impact of the enrichment process which have caused changes in the sample such as oxidation or denaturation. Nevertheless, it was important to observe with this experiment the near lack of CD3 binding on F1AA sample, containing 79.2% of BEAT″. The observed loss of CD3 binding by the BEAT″ enriched material further confirms that a modification is present on the ScFv-Fc part of the molecule.

### Cell Based Assay

In this experiment we assessed the ability of BEAT_Ab1 to activate the NFAT pathway in Jurkat cells by co-engaging CD3 and CD38 using a luciferase reporter assay. The amount of luciferase was detected by luminescence. The sample tested was enriched with about 80% of BEAT″. Binding curves and results from potency assay are shown on FIG. 13. Similarly to Biacore results, cell based assay shows a decrease in potency for all samples, with the lowest potency in the samples highly enriched in BEAT″. This confirm the data generated with Biacore and the hypothesis that the ScFv-Fc carries a modification.

### Affinity Chromatography

Next we investigated whether BEAT″ variant binds the targets CD3 and CD38 by affinity chromatography. The affinity purification was performed following the protocol supplied by ThermoFisher (reference: 20501). The AminoLink Plus Coupling Resin protocol was used. This resin allows covalent immobilization of proteins (in this study, hsCD3e and hsCD38 proteins, each in separate set) to a beaded agarose support, providing a tool for affinity purification of antibodies, antigens or other biomolecules (in this study, BEAT_Ab1). The activated support contains aldehyde functional groups that spontaneously react with primary amines on proteins or other molecules. The Schiff base bonds that form are reduced to stable secondary amine bonds in the presence of the mild reducing agent, sodium cyanoborohydride. In this study, the coupling protocols at pH 10 was used. This protocol conditions provide good immobilization yields and ligand densities. Once the ligand is immobilized, the prepared resin can be used for multiple rounds of affinity purification.

hsCD3e was produced in-house by Protein Expression department, hsCD38 is commercially available. Each target was immobilized separately on an amino coupling plus resin using 0.8 mL Pierce Centrifuge Columns and following the protocol recommended by the supplier at pH 10. BEAT_Ab1 was then purified by affinity with these two targets. Eluate was collected and analyzed by non-reducing and reducing CE-SDS.

An hsCD3e protein solution at 3.74 mg/mL (1.1 mL total) was firstly diluted 4 fold in 0.1M sodium citrate, 0.05M sodium bicarbonate, pH 10 (coupling buffer) at 0.93 mg/mL then concentrated to 450 μL using an Amicon® centrifugal unit. A final preparation of protein solution (target) at 8.8 mg/mL in 450 μL was then obtained for the immobilization step. In parallel, an hsCD38 protein solution at 5.0 mg/mL (1 mL total) was concentrated to 450 μL and the buffer was exchanged in 0.1M sodium citrate, 0.05M sodium bicarbonate, pH 10 (coupling buffer) using an Amicon® centrifugal unit. A final preparation of protein solution (target) at 8.61 mg/mL in 450 μL was then obtained for the immobilization step. A solution of BDS BEAT_Ab1 at 5.7 mg/mL (7 mL total) was concentrated to 4.5 mL, using Amicon® centrifugal units. A final preparation of the protein to purify at 7.14 mg/mL in 4.5 mL was then obtained for the purification step. During purification of BEAT_Ab1 on the column immobilized with hsCD3e, 69% of the protein to purify remained loaded in the column (5.9 mg). After washing and elution steps, a total amount of 1.48 mg (25% yield) was taken in one elution fraction (FT 2) for CE-SDS analysis, in order to identify the profile of the material which has a better affinity with hsCD3e. During purification of BEAT_Ab1 on the column immobilized with hsCD38, 51% of the protein to purify remained loaded in the column (4.4 mg). After washing and elution steps, a total amount of 1.34 mg (30% yield) was taken in one elution fraction (FT 2) for CE-SDS analysis, in order to identify the profile of the material having a better affinity with hsCD38 protein. Affinity purification using CD3 and CD38 epitopes allowed the collection of sufficient quantities of fractions binding to those molecules for CE-SDS analysis. Electropherograms from non-reduced and reduced CE-SDS are shown in FIG. 14 and FIG. 15 (in comparison to reference standard). As it can be seen, affinity purification samples confirm the findings from Biacore and potency assays and provide clear evidence that BEAT″ and a fraction of the “100 kDa species” is not binding to the CD3 epitope, while the binding to CD38 does not appear to be affected. On reduced CE-SDS performed with the same sample, it can be seen that both “Proteolytic fragment” and “Unknown” peak are not binding to CD3 epitope. A summary of results is shown in Table 4.

TABLE 4
Summary of non-reduced and reduced CE-SDS results
for affinity purification samples (ND: non detected).

| | | CD3 | CD38 | |
|---|---|---|---|---|
| Peak (% Area) | | eluate | eluate | Control |
| non- | 100 kDa species | 1.8 | 7.0 | 8.10 |
| reduced | BEAT_Ab1-BEAT′ | 1.3 | 1.7 | 2.55 |
| CE-SDS | BEAT_Ab1-BEAT | 94.9 | 86.2 | 85.96 |
| | BEAT_Ab1-BEAT″ | ND | 3.9 | 2.47 |
| reduced | Proteolytic fragment | 0.2 | 1.6 | 1.39 |
| CE-SDS | Heavy Chain | 38.1 | 39.1 | 37.69 |
| | ScFv-Fc chain | 43.4 | 38.4 | 37.63 |
| | Unknown species | ND | 2.2 | 1.59 |

### Statistical Data for Peak Assignment

From the previous experiments in enrichment and depletion (affinity purification) it appeared that there is a relationship between the content of BEAT″ and “100 kDa species” in non-reduced CE-SDS and between the content of “Unknown” and “Proteolytic fragment” peaks in reduced CE-SDS. Since a total of 9 pairs of data points was collected during enrichment and affinity purification for each of those species, the data set allowed an attempt of statistical analysis. Data was entered in JMP software (version 13.0.0). In samples where BEAT″ was not detected, it was assumed to be at zero. “Fit X by Y” analysis was performed using linear fit. The results are shown in FIG. 16—linear fit curve with confidence for fit (darker shade) and individual values (lighter shade), R² and adjusted R², and analysis of variance. The fit of BEAT″ and “Unknown” peak has an adjusted R² of 0.995 and p<0.001, while the fit of “100 kDa species” and “Proteolytic fragment” has adjusted R² of 0.985 and p<0.001.

In non-reduced CE-SDS we observed four species—“100 kDa species”, BEAT′, BEAT, and BEAT″. During reduction those species are dissociated into individual chains, which in case of fully assembled BEAT molecule are—Light Chain (LC), Heavy Chain (HC), and ScFv-Fc. For the other species observed in non-reduced CE-SDS, the following composition may be proposed based on available data (see FIG. 17 for diagrams):
100 kDa species #1—caused by proteolytic cleavage of ScFv-Fc chain above hinge region, hence lacking of CD3 binding—in reduced CE-SDS expected to be observable as: LC, proteolytic fragment (non-binding), and HC;
100 kDa species #2—caused by reduced disulphide between LC and HC and LC being dissociated in denaturing conditions of CE-SDS—in reduced CE-SDS expected to be observable as: HC and ScFv-Fc;
BEAT″—caused by a yet unidentified modification of ScFv-Fc chain which causes it to appear larger or heavier, hence enrichment by SEC and appearance of shoulder on non-reduced CE-SDS, and reduced/lack of CD3 binding—in reduced CE-SDS expected to be observable as: LC, HC, and heavier variant of ScFv-Fc.

FIG. 17 presents the different BEAT_Ab1 proposition of composition observed for the molecule during non-reduced CE-SDS (vertical) and reduced CE-SDS analysis (horizontal). Based on the CE-SDS results and knowing that the ScFv part of the BEAT_Ab1 molecule should bind to hsCD3e protein, a molecule structure for each species observed during CE-SDS analysis was proposed in FIG. 18 (non-reduced conditions) and FIG. 19 (reduced conditions). From the CE-SDS non-reduced results, it can be deduced that BEAT″ corresponds most likely to a form of BEAT_Ab1 molecule with a modification on the ScFv region, which thus modifies its affinity to hsCD3e target.

In conclusions, non-reduced and reduced CE-SDS profiles showed that BEAT″ does not have an affinity to hsCD3e, which correlates with the data generated with Biacore, potency and its proposed molecule structure (modification on the ScFv region). Moreover statistical analysis allows the identification of the peak corresponding to BEAT″ on reduced CE-SDS.

### MS Analysis—Intact Mass and Reduced

The aim of this experiment was to determine if there is real mass difference, not just difference in size (SEC—size exclusion), therefore mass determination of the native antibody chains was performed by LC-ESI-TOF-MS.

Antibody samples were separated on a C4 HPLC column (Ultimate 3000) and recorded online with a 5600 TripleTOF (AB Sciex). Before analysis 15 μl of each sample was acidified with formic acid. HPLC separation was performed on an Ultimate3000 system and subsequently fractionated using an RP-C4 column (Dr. Maisch, ReproSil Gold 300 C4). Mass spectrometry was performed on a TripleTOF 5600+ mass spectrometer (AB Sciex) operating in positive polarity mode online-coupled to the nano-LC system Mass spectrometric parameters were: mass range m/z 500-3000; Accumulation time 0.5 sec; Time bins to sum: 60; Ion spray voltage 2300 V; ion source gas 12; interface heater 70° C., alternating between CE 20 and 30. Raw data were subsequently deconvoluted using the software BioToolkit App for Peakview (AB Sciex) thus determining the protein mass.

The comparison of the bulk drug substance BEAT_AB1 BDS and the isoform enriched fraction BEAT_Ab1 F1AB allowed the determination of the intact mass of the native antibody. For both samples the expected intact mass of the native antibody (127793 Da) could be detected. For sample BEAT_Ab1 F1AB a protein mass of 130317 Da could be obtained as well. The difference of both species is 2524 Da, which can result from glycation or glycosylation, see FIG. 20 and FIG. 21.

### EXAMPLE 4: GLYCATION

Glycation is the result of the covalent binding of a sugar molecule, such as glucose or fructose, to a protein without the control of an enzyme. When antibodies are produced in a cell culture, glycation can occur following cell expression and secretion of the antibody in the culturing medium where sugars, such as glucose, are commonly present. The aim of this experiment is to induce force glycation for BEAT_Ab1 in order to verify if BEAT″ is related to glycation.

The glycation have been induced by adding a 1:1 mass ratio of glucose to the antibody solution, followed by incubation at 37° C. (after buffer exchange in PBS pH 7.4). Two different glycation have been tested in liquid and after lyophilization. Controls with the antibody have been prepared by buffer exchange in PBS at pH 7.4 incubated at 37° C. without addition of glucose. As shown in FIG. 20 to FIG. 23, glycation was induced on BEAT_Ab1, as it can be observed from the impact it had on HC and LC. Nevertheless no effect was observed on ScFv-Fc (which carry the BEAT″ modification), consequently we assumed that BEAT″ is not related to glycation.

### EXAMPLE 5: GLYCOSYLATION

Next we investigated whether BEAT″ is related to glycosylation. At this aim enzymes able to remove O-linked glycans or N-linked glycans were tested. The enzymatic treatments were performed under native and denaturing conditions, the latter allowing to explore the eventual glycosylation of hidden sites (where for instance, because of the steric hindrance, the access to the enzymes is denied). The denaturing conditions were obtained by the addition of UREA 8M (4M final) before the deglycosylation steps. Non-reduced and reduce CE-SDS analyses was performed to confirm the efficiency of enzymatic treatment

### O-Glycosylation

To investigate the presence of O-linked glycans, the following enzymes were used according to the manufacturer's instructions:
OglyZOR: an endoglycosidase that specifically hydrolyzes O-link glycans of core 1 and core 3 disaccharides on native glycoprotein (supplier GENOVIS; catalog number: G2-0G1-020);
OpeRATOR: an O-protease digesting proteins at the N-terminus of 0-glycans at serine or threonine (supplier GENOVIS; catalog number: G2-0P1-020);
SialEXO: a sialidase mix for complete removal of sialic acids on native glycoprotein (supplier GENOVIS; catalog number: G1-SM1-020).

Details of the protocols are reported in Table 5.

TABLE 5
Protocol for deglycosylation of BEAT_Ab1_BDS
using OglyZOR, OpeRATOR and SialEXO.

| | | | Reaction | | Final Enzyme |
|---|---|---|---|---|---|
| | BEAT_Ab1 | Reaction buffer | time | Temperature | Concentration |
| OglyZOR + | 400 μg | 20 mM Tris pH 6.8 | 3 h | 37° C. | 40 U/μL |
| SialEXO (unit) | | | | | |
| OpeRATOR + | 400 μg | 20 mM Tris pH 6.8 | 3 h | 37° C. | 40 U/μL |
| SialEXO (unit) | | | | | |
| SialEXO (unit) | 400 μg | 20 mM Tris pH 6.8 | 2 h | 37° C. | 40 U/μL |

### Native Conditions

The results of non-reduced CE-SDS analysis of the native BEAT_Ab1 treatment with the above-mentioned enzymes can be found in FIG. 24. No shift was observed for any of the tested enzymes, meaning that either the enzymes did not work or that no O-linked glycans are present, or that even if O-linked glycans are present, they are not of the kind removed by the tested enzymes. Additionally, BEAT″ peak was still visible. Similar results were obtained by the reduced CE-SDS analysis as shown in FIG. 25.

### Denaturing Conditions

In FIG. 26 non-reduced CE-SDS results obtained under denaturing conditions are presented. Also in this case, no deglycosylation was detected. (Additional peaks observed in samples have been identified as enzymes thanks to SDS-PAGE profiles of the enzymes used). A O-linked endoglycosidases were not able to remove BEAT″ peak, it is possible that BEAT″ did not undergo O-linked glycosylation or that the tested enzymes are not able to remove the O-linked glycans eventually present. Similar results were obtained by reduced CE-SDS analysis (FIG. 27). In this case just the treatment with enzymes OglYZOR and SialEXO are reported because Operator was found not to be compatible with denaturing conditions. The shift observed for SialEXO is due to an unknown issue during the run.

### N-Glycosylation—Glycinator and IgGZERO

It is well known that the fragment crystallizable (Fc) of an antibody bears highly conserved N-glycosylation sites, for this reasons we treated BEAT_Ab1 samples with the following enzymes able to remove Fc N-glycans, according to the manufacturer's instructions.
Glycinator: endoglycosidase able to hydrolyzes all glycoforms present at the Fc-glycosylation sites, leaving only the core GlnNac on the Fc, (supplier GENOVIS; catalog number: A0-GL8-020);
IgGZERO: IgG-specific endoglycosidase acting on complex N-glycans at the Fc-glycosylation sites leaving only the core GlnNac on the Fc, (supplier GENOVIS; catalog number: A0-IZ8-020).

Protocol used for N-deglycosylation with the mentioned enzyme can be found in Table 6.

TABLE 6
Protocol for deglycosation of BEAT_Ab1_BDS using GlycINATOR and IgGZERO.

| | | | | | Final |
|---|---|---|---|---|---|
| | | | Reaction | | Enzyme |
| | BEAT_Ab1 | Reaction buffer | time | Temperature | Concentration |
| GlycINATOR | 400 μg | 10 mM Sodium Phosphate, | 30 min | 37° C. | 40 U/μL |
| (unit) | | 150 mM NaCl pH 7.4 | | | |
| IgGZERO | 400 μg | 10 mM SodiumPhosphate, | 30 min | 37° C. | 20 U/μL |
| (unit) | | 150 mM NaCl pH 7.4 | | | |

### Native Conditions

In FIG. 28 the non-reduced CE-SDS data related to GlyciNATOR and IgGZERO treatment of BEAT_Ab1 in native conditions are shown. The enzymatic reaction clearly occurred, as demonstrated by BEAT peak shift to the left, which indicates that the molecular weight (MW) of the molecule has decreased, and therefore that deglycosylation occurred. Nevertheless, BEAT″ peak was still visible indicating that either BEAT″ is not related to a N-glycosylation or that the eventual glycosylation cannot be removed in this conditions. Reduced CE-SDS data of the sample treatment (in native conditions) by GlycINATOR and IgGZERO (FIG. 29) confirmed the results previously found by non-reduced CE-SDS.

### Denaturing Conditions

In denaturing conditions, the treatment by GlycINATOR and IgGZERO gave similar results to the ones obtained in native conditions, as shown by the non-reduced CE-SDS data in FIG. 30 and by the reduced CE-SDS analysis in FIG. 31.

### N-Glycosylation—PNGase F

The enzyme PNGase F is an amidase which cleaves between the GlcNac and asparagine residues of almost all N-linked oligosaccharides. It is a glycerol-free enzyme, therefore no glycerol used for enzymatic stability and efficiency. PNGase F (catalogue number P0704S, glycerol-free) was obtained from New England Biolabs (NEB). For BEAT_Ab1 treatment, 400 μg of BEAT_Ab1 bulk drug substance was treated by 2500 Units of PNGase F. For the denaturing conditions the antibody was incubated at 37° C. for 18 h, as specified in the NEB protocol.

### Native Conditions

The results of sample treatment with PNGase F in native conditions are shown in FIG. 32 (non-reduced CE-SDS), and in FIG. 33 (reduced CE-SDS). In both the cases, the observed BEAT peak shift to the left indicates that deglycosylation by PNGase F enzyme occurred, nevertheless BEAT″ peak was still present.

### Denaturing Conditions

The treatment of BEAT_Ab1 with PNGase F in denaturing conditions was useful to verify the presence of N-glycans in non-consensus sites. In fact, according to Valliere-Douglass J. F., et al 2010. J. Biol. Chem. 285: 16012-16022, non-consensus N-linked glycans can be removed by PNGase F under denaturing condition.

FIG. 34 shows reduced CE-SDS data where PNGase F treatment leads to the complete disappearing of the shoulder after ScFv-Fc peak. Given that denaturing condition themselves do not have an impact on BEAT″ shoulder, we can conclude that the removal of the shoulder is related to the use of the PNGase F and not denaturing conditions, and that BEAT″ carries a N-linked glycan present on a non-consensus glycosylation sites.

In order to verify the effect of PNGase F under denaturing condition, a spiking experiment was performed using enriched BEAT″ material. For this 50% of enriched material in BEAT″ (80% enriched) and 50% of BEAT_Ab1_BDS have been mixed together. PNGase F under denaturing condition have been tested on this sample to check if this enzyme could remove a high level of BEAT″. In FIG. 35 we can see that BEAT″ is still visible for the spiked sample and control condition (BEAT_Ab1_BDS). But for the spiked sample treated with PNGase F under denaturing condition we can see a total removal of BEAT″.

Additionally the enzyme rapid PNGase was tested in both reducing and non-reducing conditions. The results, shown in FIG. 36, indicate that deglycosylation occurs (mass shift to the left) and we can also see that BEAT″ has been efficiently removed.

The obtained results indicate that BEAT″ is related to a non-consensus glycosylation and that it can be efficiently removed using PNGase F (denaturing protocol) and rapid PNGase F (both reducing and non-reducing format).

### EXAMPLE 6: CHARACTERIZATION OF THE NON-CONSENSUS GLYCOSYLATION VARIANTS

Having established that BEAT″ has a non-consensus glycosylation present on the ScFv part of the BEAT_Ab1 molecule, we next decided to identify non-consensus glycosylation sites on BEAT_Ab1 ScFv chain and to determine which glycan structures are present. We first looked at the protein sequence for consensus glycosylation sites (N—X—S/T), and non-consensus sites—“reverse consensus” (S/T—X—N), and “glutamine-linked” (QGT).

### Material and Method:

### 1.1 Peptide Mapping

### 1.1.1 Sample Preparation

For both samples, an aliquot of the sample solution equivalent to 100 μg protein was buffer exchanged against freshly prepared 6 M Guanidine hydrochloride, 25 mM Ammonium bicarbonate solution using Zeba spin desalting columns (0.5 ml, 7K MWCO). Buffer-exchanged sample solutions were reduced with 5 mM TCEP for 1 hour at 60° C. Reduced sample solutions were alkylated with 15 mM IAA for 30 minutes at room temperature and protected from light. Excess of IAA was then quenched through addition of 10 mM DTT. Aliquots of alkylated sample solutions were buffer-exchanged against 100 mM Tris-HCl, 1 M Urea, 10 mM CaCl2), 20 mM Methylamine using Zeba spin ating columns (0.5 ml, 7K MWCO). Alkylated and buffer-exchanged samples were simultaneously digested with Lys-C (Promega, Mass spectrometry grade) and Trypsin (Promega, Sequencing grade modified trypsin) for 4 hours at 37° C. (weight-to-weight Lys-C/Trypsin/substrate ratio of 1/2/50).

### 1.1.2 Sample Analysis

UPLC-UV-MSE analyses were performed using a Waters Acquity UPLC H-Class integrated system coupled to a Waters Synapt G2-Si HDMS Q-Tof (UGA579) mass spectrometer. Calibration of the mass spectrometer was performed using Sodium Iodide. Mass accuracy was better than 5 ppm for the major m/z signals observed prior to sample. In addition, Leu-Enkephaline solution was regularly sprayed into the source of the instrument to allow real time mass correction during the acquisition (Lockspray). Aliquots of sample solutions were injected on a C18 reversed phase column connected to the source of the mass spectrometer and analyzed using the conditions described below.

### 1.1.2.1 UPLC Conditions

Autosampler temperature: set at 8° C.;

### Solvent A: 0.05% Formic Acid in Water;

### Solvent B: 0.05% Formic Acid in 90% ACN/10% Water (v:v);

Column: Waters Acquity UPLC BEH C18, 1.7 μm, 150 mm×2.1 mm;
Flow rate: set at 400 μL/min;
Column temperature: set at 60° C.;
UV detection: 214 nm and 280 nm;
Injection volume: 1 μL.

### 1.1.3 Data Processing and Interpretation

UPLC-UV-MSE data were acquired and processed using MassLynx™ software version 4.1. Interpretation of the raw data was aided by the use of the BioLynx™ software supplied with the current version of MassLynx™ and the protein sequence. To determine the nature of product related impurity, targeted data interpretation was oriented towards possible presence of non-consensus glycosylation located on “QGT” sequence of scFv-Fc chain. scFv-Fc chain contains two “QGT” sequences, localized within Trypsin/Lys-C peptides 101-158 and 205-246.

### 1.2 N-Glycan Profiling

### 1.2.1 Sample Preparation

A 100 μg sample aliquot was subjected to EndoS treatment at room temperature for 15 min using deGlyclT™ Microspin column (Genovis), following Supplier's protocol. Sample aliquots, corresponding to 100 μg, were subjected to reduction using DTT for 1 h at 45° C. then to alkylation using IAM for 30 min at room temperature in the dark. The reduced and alkylated samples were buffer-exchanged against 50 mM Ammonium bicarbonate solution using a 3 kDa MWCO Amicon centrifugal device before being subjected to digestion with trypsin (ratio enzyme:sample 1:50, 37° C., 6 hours). The digestion was stopped by submitting sample solution to a temperature of 100° C. for 3 min. The resulting peptide/glycopeptides mixtures were treated with PNGase F (Roche) for approximately 20 h at 37° C. Released N-glycans were purified using a C18 Sep-Pak cartridge before being dried-down using a rotative evaporator. Purified N-glycans were permethylated using DMSO, NaOH and ICH3 then extracted in chloroform and purified using a SepPak C18 cartridge before being dried-down using a rotative evaporator.

### 1.2.2 Sample Analysis

Analyses were performed on a Sciex 5800 MALDI-TOF/TOF mass spectrometer. The instrument was calibrated using the Sciex calibration mixture prior to analyses. Mass accuracy was better than ±0.2 m/z for the major signals observed prior to sample analysis. A solution of 2,5-dihydroxybenzoic acid matrix (DHB) at 20 mg/mL was prepared in MeOH: 0.1% aq TFA (v:v 1:1). Permethylated N-glycans were resuspended in MeOH, mixed with an equal volume of DHB matrix then spotted onto a MALDI target plate and left to dry at RT. Samples were analysed in Reflectron Positive ion mode over the m/z range 500 to 5000. MALDI-MS spectra from 2′000 shots were summed. The major molecular ions attributed to glycans were selected for MS/MS fragmentation analyses using air as collision gas. MALDI MS/MS spectra from 4′000 shots were summed. MALDI-TOF data were acquired using 4000 Series Explorer™ software version 4.1.0.

### 1.2.3 Data Processing and Interpretation

Raw data were processed using Data Explorer version 4.11 (built 125). Only signals with a relative intensity above 5% of major signal were reported.

### Results:

### Site Identification:

Differential EIC-based profiling demonstrated that PNGase F treatment specifically increased deamidation of ScFv-Fc peptide 101-158 in impurity-enriched sample (FIG. 37C and FIG. 37D), but not in control sample (FIG. 37A and FIG. 37B). To support this data, targeted search was carried out to determine the presence of ScFv-Fc peptide 101-158 bearing G2FS2 glycosylation, which was the major N-glycan form observed in glycan profiling of EP180/BEAT″ enriched sample. Glycopeptide-specific EICs obtained for EP180/BEAT″ sample without PNGase F treatment allowed to evidence a signal corresponding to glycosylated scFv-Fc peptide 101-158 (FIG. 38C). It should be noted that minor signal assigned to G2FS2 glycosylated scFv-Fc peptide 101-158 was also detected in BEAT_Ab1 (FIG. 38A). Of note, these signals were no longer observed following PNGase F treatment, confirming signals assignment to glycosylated peptide (FIG. 38B and FIG. 38D).

For the second site the results show no deamidation induced by PNGase F digestion suggest that the peptide containing the “QGT” motive is not modified by glycosylation. (Data not shown)

### N-Glycan Profiling

### Control Sample:

The N-glycan population of BEAT_Ab1 control sample was determined by release of the N-glycans using PNGase F, purification, permethylation and MALDI-TOF MS analysis. Data generated are summarized in FIG. 39. Structural assignments were deduced from monosaccharide composition calculated from measured molecular weight, MS/MS fragmentation patterns and knowledge of the glycan biosynthetic pathways. A series of singly charged [M+Na]+ ions consistent with a homogeneous population of complextype N-glycans was detected. The spectrum is composed of major molecular ions consistent with G0F (m/z 1836), followed by signals corresponding to G1F and G2F (m/z 2040 and 2244, respectively). N-glycan structures are summarized in FIG. 39 and MS/MS fragmentation spectra are presented in FIG. 40 to FIG. 42.

### BEAT″ Enriched Sample

Taking advantage of the resistance of non-consensus N-glycosylation sites towards EndoS treatment, N-glycan profiling was performed on EndoS-digested impurity-enriched sample. This strategy offered the advantage of profiling specifically EP180/BEAT″ enriched non-consensus N-glycosylation sites. Data generated are summarized in FIG. 43. Structural assignments were deduced from monosaccharide composition calculated from measured molecular weight, MS/MS fragmentation patterns and knowledge of the glycan biosynthetic pathways. A series of singly charged [M+Na]+ ions consistent with a heterogeneous population of complex-type N-glycans was detected. Major signal corresponds to core fucosylated biantennary disialylated structure (G2FS2) contrasting with major neutral N-glycans G0F and G1F observed at consensual N-Glycosylation sites of BEAT_Ab1-BDS sample. N-glycan structures are summarized in Table 15 and MS/MS fragmentation spectra are presented in FIG. 44 and FIG. 45.

### Conclusions

The glycated variant BEAT_Ab1 BEAT″ is located on the ScFv part of the molecule on the first QGT site present on the peptide 101-158. The ScFv-Fc sequence can be found in FIG. 46 with highlighted glycosylation site with different color depending of their nature. The results given by the N-glycan profiling show two potential glycated structure for BEAT″ as shown in FIG. 47. Glycans detected at QGT site were found to be same structures as fount on Fc part (FIG. 44)—G0F and G1F, plus mono-sialylated variant of G1F and G2F, and di-sialylated variant of G2F.

### EXAMPLE 7: BEAT″ ENRICHMENT BY CEX

Because of the finding of BEAT″ sialylation, which makes this species more acidic than BEAT variant, we decided to investigate BEAT″ enrichment by CEX-HPLC. The aim of this experiment was to separate BEAT_Ab1 species using CEX-HPLC according to their charges to isolate a fraction with an enriched level in BEAT″.

This experiment was performed using charrette HPLC system with fraction collector.

The column used was ProPac WCX-10, BioLC, Semi-prep 9×25 mm.

The eluent used was: (see logbook solutions record #06 for more details)
Eluent A: 20 mM NaPhosphate, pH 6.5;
Eluent B: 20 mM NaPhosphate, 100 mM NaCl, pH 6.9.

FIG. 48 shows the standard CEX profile of BEAT_Ab1 and the peaks collected during this first experiment. After the collection the fractions have been concentrated and desalted and then analyzed by capillary gel electrophoresis under reduced and non-reduced conditions.

In FIG. 49 and FIG. 50 we can see an overlay of the 7 collected fractions with the non-fractionated starting material respectively for non-reduced and reduced cGE. For the first three fractions F1 to F3 corresponding to the more acidic species some parts of the molecules have been enriched. For the other acidic fractions F4 and F5, main peak (F6) and basic peak (F7) it seems that there is no difference compared to the starting material. For BEAT″ there is a huge level of enrichment in F1 and F2. This species is slightly enriched in F3 and no more enrichment is visible for all the other fractions.

TABLE 7
Non-reduced (NR) CE-SDS data for the peak of interest

| cGE NR | STD | F1 | F2 | F3 | F4 | F5 | F6 | F7 |
|---|---|---|---|---|---|---|---|---|
| 100 kDa | 7.152 | 20.875 | 58.373 | 14.596 | 7.551 | 9.184 | 5.72 | 10.066 |
| BEAT′ NR | 1.431 | 4.304 | 2.27 | 2.797 | 3.627 | 4.512 | 3.09 | 4.864 |
| BEAT″ NR | 3.707 | 46.066 | 15.684 | 6.5 | 0 | 0 | 0 | 0 |

TABLE 8
Reduced (R) CE-SDS data for the peak of interest

| cGE R | STD | F1 | F2 | F3 | F4 | F5 | F6 | F7 |
|---|---|---|---|---|---|---|---|---|
| Fragment | 1.386 | 6.305 | 16.201 | 2.398 | 0 | 0 | 0 | 0 |
| BEAT″ R | 1.593 | 20.228 | 7.431 | 8.3 | 0.673 | 0 | 0 | 0 |

1. A purified antibody or fragment thereof comprising a single chain variable fragment which is glycosylated in at least one non-consensus glycosylation site.

2. The purified antibody or fragment thereof of claim 1, wherein said purified antibody or fragment thereof binds CD3.

3. The purified antibody or fragment thereof of claim 1, wherein said single chain variable fragment comprises a variable heavy chain having an amino acid sequence selected from the group consisting of SEQ ID NOs: 1, 2 and 3, and conservative modifications thereof, and a variable light chain having an amino acid sequence selected from the group consisting of SEQ ID NOs: 4, 5 and 6, and conservative modifications thereof.

4. The purified antibody or fragment thereof of any one of the preceding claims, wherein said non-consensus glycosylation site is a QGT motif.

5. The purified antibody or fragment thereof of claim 1, wherein said non-consensus glycosylation site is glycosylated with a glycan selected from the group consisting of G0F, G1G, G1FS, G2FS and GSFS2.

6. The purified antibody or fragment thereof of claim 4, wherein said single chain variable fragment is glycosylated in a QGT motif at positions 117-119 of SEQ ID NO: 2 with a glycan selected from the group consisting of G2FS and G2FS2.

7. The purified antibody or fragment thereof of claim 1 comprising the amino acid sequences of SEQ ID NOs: 7, 8 and 9 or the amino acid sequences of SEQ ID NOs: 10, 11 and 12.

8.-14. (canceled)
